SEQUENCE LISTING (24 sequences)

SEQ ID NO: 1 - DNA, 29 bp, Synthetic construct
  source 1..29: mol_type = other DNA
ggtccaccaaacgtaatgcggggtaaaaa

SEQ ID NO: 2 - DNA, 42 bp, Synthetic construct
  source 1..42: mol_type = other DNA
aaaaaaaaaaaaaaaaaaaacactgcgttctccattctggtt

SEQ ID NO: 3 - DNA, 66 bp, Synthetic construct
  source 1..66: mol_type = other DNA
ggtccaccaaacgtaatgcggggtaaaaaaaaaaaaaaaaaaaaactgagtccagctcgaaactta

SEQ ID NO: 4 - DNA, 66 bp, Synthetic construct
  source 1..66: mol_type = other DNA
ggtccaccaaacgtaatgcggggtaaaaaaaaaaaaaaaaaaaatgtcgacgaggtccgagtgtaa

SEQ ID NO: 5 - DNA, 64 bp, Synthetic construct
  source 1..64: mol_type = other DNA
ggtccaccaaacgtaatgcggggtaaaaaaaaaaaaaaaaaaaagtatctcttcatagccttat

SEQ ID NO: 6 - RNA, 48 nt, SARS-CoV-2 Virus
  source 1..48: mol_type = genomic RNA
accccgcattacgtttggtggaccctaaccagaatggagaacgcagtg

SEQ ID NO: 7 - RNA, 1362 nt, SARS-CoV-2 Virus
  source 1..1362: mol_type = genomic RNA
aattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagaca

SEQ ID NO: 8 - DNA, 28 bp, Synthetic construct
  source 1..28: mol_type = other DNA
agagatcatttaatttagtaggaaaaaa

SEQ ID NO: 9 - DNA, 41 bp, Synthetic construct
  source 1..41: mol_type = other DNA
aaaaaaaaaaaaaaaaaaaacaagctataacgcagcctgta

SEQ ID NO: 10 - DNA, 65 bp, Synthetic construct
  source 1..65: mol_type = other DNA
agagatcatttaatttagtaggaaaaaaaaaaaaaaaaaaaaaactgagtccagctcgaaactta

SEQ ID NO: 11 - DNA, 65 bp, Synthetic construct
  source 1..65: mol_type = other DNA
agagatcatttaatttagtaggaaaaaaaaaaaaaaaaaaaaatgtcgacgaggtccgagtgtaa

SEQ ID NO: 12 - DNA, 63 bp, Synthetic construct
  source 1..63: mol_type = other DNA
agagatcatttaatttagtaggaaaaaaaaaaaaaaaaaaaaagtatctcttcatagccttat

SEQ ID NO: 13 - RNA, 46 nt, SARS-CoV-2 Virus
  source 1..46: mol_type = genomic RNA
cctactaaattaaatgatctctgaatacaggctgcgttatagcttg

SEQ ID NO: 14 - DNA, 30 bp, Synthetic construct
  source 1..30: mol_type = other DNA
actatcatcatctaaccaatcttcaaaaaa

SEQ ID NO: 15 - DNA, 45 bp, Synthetic construct
  source 1..45: mol_type = other DNA
aaaaaaaaaaaaaaaaaaaattccatctctaattgaggttgaacc

SEQ ID NO: 16 - DNA, 66 bp, Synthetic construct
  source 1..66: mol_type = other DNA
actatcatcatctaaccaatcttcaaaaaaaaaaaaaaaaaaaaactgagtccagctcgaaactta

SEQ ID NO: 17 - DNA, 66 bp, Synthetic construct
  source 1..66: mol_type = other DNA
actatcatcatctaaccaatcttcaaaaaaaaaaaaaaaaaaaatgtcgacgaggtccgagtgtaa

SEQ ID NO: 18 - DNA, 64 bp, Synthetic construct
  source 1..64: mol_type = other DNA
actatcatcatctaaccaatcttcaaaaaaaaaaaaaaaaaaaagtatctcttcatagccttat

SEQ ID NO: 19 - DNA, 27 bp, Synthetic construct
  source 1..27: mol_type = other DNA
aaaaataagtttcgagctggactcagt

SEQ ID NO: 20 - DNA, 27 bp, Synthetic construct
  source 1..27: mol_type = other DNA
aaaaattacactcggacctcgtcgaca

SEQ ID NO: 21 - DNA, 25 bp, Synthetic construct
  source 1..25: mol_type = other DNA
aaaaaataaggctatgaagagatac

SEQ ID NO: 22 - RNA, 53 nt, SARS-CoV-2 Virus
  source 1..53: mol_type = genomic RNA
agaagattggttagatgatgatagtaaaggttcaacctcaattagagatggaa

SEQ ID NO: 23 - RNA, 163 nt, SARS-CoV-2 Virus
  source 1..163: mol_type = transcribed RNA
caaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtctt

SEQ ID NO: 24 - RNA, 1363 nt, SARS-CoV-2 Virus
  source 1..1363: mol_type = transcribed RNA
aattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacaa

(19) United States
(12) Patent Application Publication
(10) Pub. No.: US 2026/0201447 A1
(43) Pub. Date: Jul. 16, 2026

(54) KIT AND PROCESS FOR THE DETECTION OF A TARGET NUCLEIC ACID

(71) Applicants: UNIVERSITE DE STRASBOURG, STRASBOURG (FR); CENTRE NATIONAL DE LA RECHERCHE SCIENTIFIQUE, PARIS (FR)

(72) Inventors: Andrey KLYMCHENKO, ILLKIRCH-GRAFFENSTADEN (FR); Elisabete CRUZ DA SILVA, STRASBOURG (FR); Paraskevi GAKI, ILLKIRCH-GRAFFENSTADEN (FR); Andreas REISCH, COLMAR (FR); Sébastien PFEFFER, STRASBOURG (FR)

(73) Assignees: UNIVERSITE DE STRASBOURG, STRASBOURG (FR); CENTRE NATIONAL DE LA RECHERCHE SCIENTIFIQUE, PARIS (FR)

(21) Appl. No.: 19/123,145

(22) PCT Filed: Oct. 27, 2023
(86) PCT No.: PCT/EP2023/080064

§ 371 Date: Apr. 22, 2025

(30) Foreign Application Priority Data
Oct. 27, 2022 (EP) 22306627.5

(51) Int. Cl. C12Q 1/6825; C12Q 1/6806; C12Q 1/6834; G01N 21/64

(52) U.S. Cl. CPC C12Q 1/6825; C12Q 1/6806; C12Q 1/6834; G01N 21/6428; G01N 2021/6439

(57) ABSTRACT

A kit and a sandwich type process using this kit, for detecting with high sensitivity and selectivity a target nucleic acid and use of the kit or process for the detection of a target nucleic acid and for the diagnosis of a disease or a condition. The kit and a method are particularly useful for detecting point mutation on a target nucleic acid.

### DOMAIN OF THE INVENTION

The subject of the invention is a kit and a sandwich type process using said kit for the sensitive and robust detection of a target nucleic acid in a sample. The invention also relates to the use of a kit or a process according to the invention for example for the molecular diagnosis of a disease or a condition. The invention pertains to the domain of molecular biology assays.

### BACKGROUND OF THE INVENTION

Nucleic acids are considered as important markers of diseases such as cancer or infections, however their detection may be difficult due to their presence at very low concentration.

Disease-relevant nucleic acid biomarkers are often present in an ultralow abundance, thus precise and robust detection of analytes mainly in bulk biofluids is of great importance for biological research, precision medicine and early-stage diagnosis. COVID-19 pandemic uncovered the critical need for rapid, highly sensitive and specific detection methods for controlling the rapidly evolving pandemics. In clinical diagnosis, direct severe acute respiratory syndrome coronavirus 2 (SARS-COV-2) nucleic acid testing has not been applied due to challenging detection of ultralow concentrations of RNA molecules in suspension. The direct detection is limited by strong background coming from non-specific proteins, nucleic acids or other biomolecules.

In molecular diagnostics, there are two types of assays with specific advantages each: (1) target amplification and (2) signal amplification assays. Target amplification assays, such as PCR, are sensitive, but require target extraction and purification, enzymatic reactions, and are more prone to false-positives. Contrarily, signal amplification assays are simpler since they tend to skip nucleic acid purification and enzymatic amplification. Thus, there is less loss of material and lower rate of false positives. However, compared to PCR-based target amplification method, signal amplification assays are less sensitive. Many efforts have been devoted to the development of ultrasensitive signal amplification assays to detect unamplified nucleic acids. A potential portable microfluidic device showed specific and sensitive detection of Ebola nucleic acids compared to RT-PCR (Cai, H. et al., 2015). Interestingly, some of these works were able to directly detect target nucleic acids from whole blood lysates (Ngo, H. T. et al., 2018) (Zheng, Z. et al., 2006).

To date, the recommended methodology for COVID-19 diagnostics is based on quantitative reverse-transcription polymerase chain reaction (qRT-PCR) that requires nucleic acid extraction from a pharyngeal swab and target amplification procedures. Such procedures need skilled personal, specific equipment, and long processing times (>2 h), making on-site SARS-COV-2 nucleic acid testing difficult to put in place. Moreover, the high demand for commercial RNA extraction kits led to a shortage of these reagents and thus several diagnostic workflows overpassing the long intermediate RNA extraction step were developed. In the last decade, several studies showed the performance of magnetic beads in the extraction of nucleic acids from biofluids prior to molecular detection. Magnetic beads are composed of iron oxide nanoparticles embedded in a polymeric matrix and can isolate nucleic acids differently according to surface functionalization. This functionalization affects binding kinetics and compatibility with molecular detection strategies. For example, silica-coated beads bind non-selectively to all nucleic acids via electrostatic interactions, so they are mainly used with detection methods tolerant to high concentrations of non-target background nucleic acids, like RT-PCR. Oligonucleotides-coupled beads, such as oligo (dT) beads or beads surface functionalized with specific sequences, are used for mRNA targets extraction and have high recovery rates. This purification can be used either manually, within a microfluidic chips or in an automated robotic setup. Besides isolation and elution of nucleic acids, several specific nucleic acid sequences have been detected directly on beads surface by hybridization assays using different materials (e.g. biotin-avidin, protein-enzyme, fluorescent dyes, quantum dots, etc. . . . ). However, most of these methods are limited by low signal intensity or rapid photobleaching (Lim et al., 2009).

EP 3 536 806 “Oligonucleotide-functionalized hydrophobic polymer nanoparticles” describes nanoparticles comprising a hydrophobic polymer and luminescent components, as energy donor elements, that can be used to detect target nucleic acids. Said nanoparticles can be coupled to oligonucleotides, either complementary to a specific target or a non-specific sequence of a target. Target-specific oligonucleotides are conjugated to energy-donating elements and their nucleotide sequence is complementary to another nucleotide sequence labeled with an excitation energy acceptor compound. A FRET (Fluorescence Resonance Energy Transfer) effect occurs between the acceptor compound and the donor compound of said nanoparticles. The lower limit of detection using these nanoparticles in the described process is a concentration of 5 μM of target nucleotide.

WO 2017/220453 describes the detection of mutations using magnetic beads. A magnetic bead-bound probe hybridizes with one end of a target nucleic acid and a surface bound probe hybridizes with the other end of said target nucleic acid. Stringency is applied on hybridization complexes via magnetic force and/or temperature.

There is still a need for a simple, rapid, sensitive and robust process for the detection for the ultra-low abundant nucleic acids.

### SUMMARY OF THE INVENTION

In the present work, the inventors aimed to develop a sandwich hybridization process for the detection of low abundant nucleic acids molecules from a sample. A process according to the invention involves hybridization in which the nucleic acid of interest is detected by dual direct hybridization using different complementary oligonucleotides that bind specifically to different regions of the target nucleic acid.

A process according to the invention combines:
the capture of target nucleic acid molecules using solid surface-based hybridization of sequence-specific probes to target in solution, wherein capture probes are immobilized on a solid substrate, to allow isolation and enrichment of the target molecules directly from raw samples with minimal processing, and
at least a second-sequence specific capture allowing direct detection on said solid surface through binding to ultrabright luminescent particles.

The capture of target nucleic acid molecules using solid surface-based hybridization involves a solid surface, which can be either an immobilized surface or the surface of a solid particle such as a bead, for example a magnetic or a glass bead. The isolation of a complex bead/target is achievable for example by applying a magnetic force or gravity, centrifugation or filtration, depending on the nature of said solid surface. This isolation is important to remove the excess of non-bound DNA modified nanoprobes and/or ultrabright luminescent particles. It can be useful to separate said target nucleic acid of interest even from a cell lysate without requiring RNA extraction. The detection of the fluorescence signal occurs directly on the solid surface by fluorescence quantification, as the fluorescent particle binds through complementary hybridization to the target.

A kit and a process according to the invention are useful for detection of a target nucleic acid in a sample, such as an environmental sample or a biological sample. A kit and a process according to the invention are also useful for diagnostic applications.

The advantages of a kit and a process according to the invention are their extremely high sensitivity, their robustness and simplicity, the fact that it is an enzyme-free amplified detection assay.

Indeed, a kit and a process according to the invention allow a precise and robust detection of nucleic acids with limit of detection around 1 fM. Extreme brightness of luminescent particles is critical to achieve high signal to noise ratio for rapid detection of the target by simple techniques.

Furthermore, low abundant target nucleic acid molecules can be detected and isolated without requiring a supplementary nucleic acid extraction step, as do detection kits and methods according to prior art.

Target nucleic acid molecule may be any nucleic acid molecule, including double-stranded and single-stranded DNA molecules and double-stranded and single stranded RNA molecules, and including short size and long-size nucleic acid molecules. Point-mutations are detectable using a kit or a method of the invention.

Ultrabright particles brightness and the high concentration of complementary oligonucleotides to be attached to the beads and to the ultrabright luminescent particle, are great advantages of a kit and a test of the present invention, to improve the limit of detection of nucleic acid. Indeed, the limit of detection of a assay according to the invention is around 1 fM, using a fluorescence plate reader. This represents a dramatic improvement of the limit of detection compared to prior art tests. A kit and a test according to the present invention enable the detection of RNA and DNA of different lengths and can be used for the detection of nucleic acids in samples. For example, a kit and a test according to the present invention can be used for the diagnosis of diseases or for the detection of a pathogen, such as a parasite, in an environmental sample.

The invention makes it possible to carry out, in a limited number of steps, both steps of detection of target nucleic acid and extraction of said target nucleic acid from its original medium. The proposed test is very sensitive, as its detection limit is around 1 fM. The invention does not require amplification, in particular by PCR, nor the use of enzymes. The invention makes it possible to detect nucleic acids from short size microRNAs to long size nucleic acids, using the direct detection of a luminescent signal.

Finally, the fluorescence signal generated with a test according to the invention can be read by using a plate reader, which accelerates the detection of nucleic acids in large scale samples.

In a particular embodiment, a process according to the invention takes advantage of the synergy of combined capture with beads-based, and particularly magnetic beads-based, solid-surface hybridization and of ultrabright fluorescent particles.

In another particular embodiment, a kit and a process according to the invention take advantage of the synergy of combined capture with solid-surface based hybridization and detection by using ultrabright fluorescent DNA-nanoparticles (DNA NPs) such as disclosed in WO 2017/220453.

In a first aspect, the present invention relates to a kit for the sandwich-test detection of a target nucleic acid in a sample. In a second aspect, the present invention relates to a process using a kit of the invention for the sandwich-test detection of a target nucleic acid in a sample.

### DETAILED DESCRIPTION OF THE INVENTION

In a first aspect, the present invention relates to a kit for the detection of a target nucleic acid in a sample, wherein said kit comprises at least:
i) a probe P1 comprising, or consisting of, a nucleic acid fragment NA1 linked to a functional unit F1, wherein NA1 comprises or consists of a nucleotide sequence complementary to the nucleotide sequence of a region T1 of the target nucleic acid,
ii) a functional unit F2 bound to a solid surface, wherein F2 is exhibiting a high affinity for F1 or is covalently bound to F1,
iii) a probe P2 comprising, or consisting of, a nucleic acid fragment NA2 linked to an ultrabright luminescent particle exhibiting a brightness of at least 10⁷ M⁻¹ cm⁻¹, preferably at least 2×10⁷ M⁻¹ cm⁻¹, at least 5×10⁷ M⁻¹ cm⁻¹, at least 10×10⁷ M⁻¹ cm⁻¹ or at least 40×10⁷ M⁻¹ cm⁻¹.

A non-limiting illustration of the elements of a kit according to the invention, and of their respective interactions is shown in FIG. 1A.

By “kit” is meant a kit comprising two or more separate components that are contained together in conventional material, such as a package. These separate components interact for a specific purpose for achieving a specific result. It is to be understood that the term “comprising” is not limiting. The term “consisting of” is a considered preferred embodiment of the term “comprising”.

The term “sample” refers to any small quantity of a medium susceptible to contain a target nucleic acid. Among samples, one can cite biological samples, comprising cells or tissues of any origin. One can also cite environmental samples, provided from extraction or removal from the environmental matrix, such as water for example.

In a particular embodiment, the present invention relates to a kit for the in vitro detection of a target nucleic acid in a sample.

The expression “in vitro” has its conventional meaning and designates experiments performed on biological molecules or living cells which have been extracted from their normal environment and are provided as samples, to be analysed.

The term “nucleic acid” refers to a deoxyribonucleotide or a ribonucleotide polymer, and encompasses natural nucleotides and known analogues of natural nucleotides that can function in a similar manner as naturally occurring nucleotides. Non-limiting examples of said analogues of natural nucleotides are locked nucleic acids (LNA) and 2′-O-methylated nucleotides. A target nucleic acid molecule detected by a kit or a method according to the invention is any nucleic acid molecule, including single-stranded and double-stranded DNA molecules and single stranded and double-stranded RNA molecules, including microRNA molecules.

A nucleic acid fragment of probe P1 or probe P2 is preferably an oligonucleotide, by “oligonucleotide” is meant a nucleic acid comprising, or consisting of, a sequence of at least 5 nucleotides, preferably comprising, or consisting of, a sequence of at least 8, 9, 10, 11, 12, 13, 14, 15, 16, 17, 18, 19, or at least 20 to at least 150 nucleotides.

“Complementary” means that said nucleic acid sequences are able to form a stable hybridization complex upon appropriate stringency conditions. Sequence-specific hybridization uses oligonucleotide probes complementary to the sequence of interest, capturing it by hybridization. “Hybridization” means the formation of a stable complex between complementary nucleic acid molecules. Hybridization may occur between partially complementary molecules and between totally complementary molecules. The stability of the formed hybridization complex relies on the reaction conditions and on the proportion of complementary nucleic acid sequences within nucleic acid molecules. The sequence of the oligonucleotides is designed in order to ensure the formation of a stable hybridization complex.

It is preferred that the length of a nucleotide sequence of a probe P1, P2 or P3 complementary to a nucleic acid is of at least 8 nucleotides, preferably at least 8, 9, 10, 11, 12, 13, 14, 15, 16, 17, 18, 19, or at least 20 to at least 150 nucleotides.

Within probe P1, the nucleotide acid fragment NA1 is bound to a functional unit F1 through a covalent or a noncovalent bond, and preferably through a covalent bond. Within probe P2, the nucleotide acid fragment NA2 is bound to an Ultrabright Luminescent Particle (ULP) through a covalent or a noncovalent bond, and preferably through a covalent bond.

In a particular embodiment of a kit according to the invention, a probe P1 comprises, or consists of: a nucleic acid fragment NA1 and a functional unit F1, wherein NA1 and F1 are bound via a linker. In another particular embodiment of a kit according to the invention, a probe P2 comprises, or consists of: a nucleic acid fragment NA2 and a ULP, wherein NA2 and ULP are bound via a linker.

By “linker” it is intended any appropriate compound chosen by a person skilled in the art and comprising:
a non-coding nucleotide sequence chosen among DNA linker, such as for example a “A10” or a “A20” linker (if present said DNA linker comprises nucleotides that do not take part to the complementary hybridized sequences) and/or
a non-nucleic element, such as for example a chemical element, such as, for tetraethylene glycol (TEG).

By “functional unit”, it is intended any molecule, biomolecule or chemical group, capable to form a non-covalent or a covalent bond selectively with another functional unit.

Among functional units commonly used by a person skilled in the art, one can cite biotin (BIO), avidin (A), neutravidin (N) and streptavidin (SV). Biotin is known for its very high affinity for avidin, neutravidin and streptavidin. Functional units can be also complementary nucleic acid sequences of at least 12 bases.

In a particular embodiment, the first functional unit is biotin (BIO), wherein the second functional unit is chosen among the group consisting of streptavidin (SV) and streptavidin analogues, in particular among avidin (A) and neutravidin (N).

In a first embodiment of the invention, F2 is linked through a non-covalent bond to F1 and exhibits a high affinity for F1. By “non-covalent bond” is meant a bond that does not involve the sharing of electrons and rather involves other electromagnetic interactions. Non covalent bonds include hydrophobic effects, van der Waals force, electrostatics and Pi-effects. Among non-covalent bonds, one can cite for example non-covalent bonds n between complementary nucleic acid molecules. By “high affinity” is meant a binding complex affinity characterized by a kD of at least 1 nM (upper limit), preferably of at least 1 pM. In a particular example, the first functional unit is biotin (BIO), the second functional unit is streptavidin, neutravidin or avidin. In this embodiment, F1 and F2 are capable to form together a binding complex of a high affinity through a non-covalent (without sharing of electrons) interaction. The very high affinity between, for example, biotin and streptavidin, allows the formation of a stable non-covalent complex, this stability is observed even when an external physical force is applied on said complex. In a more particular embodiment, the functional unit F1 is biotin and the functional unit F2 is avidin or streptavidin.

In a second embodiment of the invention, F2 is linked via a covalent bound to F1. By “covalent bond”, it is intended a chemical bond wherein electrons are shared and form at least one pair of electrons between two atoms. In case of a covalent bond, functional units are chemically reactive groups. In a particular embodiment, functional group can be azide, which can react with derivatives of acetylene by “click” cycloaddition reaction. In a particular embodiment, functional group can be carboxylate that can react with amine forming covalent amide bond.

In a kit or process according to the invention, F1 is bound covalently or non-covalently to a nucleic acid fragment NA1 and is able to bind covalently or non-covalently to F2, and F2.

The functional unit F2 is bound to a solid surface by any technique known by a person skilled in the art, through either a covalent or a non-covalent bond. For example, to functionalize a glass surface with streptavidin, an established approach is based on adsorption of BSA-biotin, further binding of streptavidin and finally addition of corresponding DNA capture bearing biotin unit is used (FIG. 2A).

In first particular embodiment of a kit according to the invention, a functional unit F2 is bound to a solid immobilized surface, such as: a glass surface, the surface of a reaction support, the surface of a multi-well plate, the surface of a microfluidic device or any surface appropriate for covalent or non-covalent functionalization.

In second particular embodiment of a kit according to the invention, a functional unit F2 is bound to a surface which is the surface of a solid particle. This solid particle can be for example a magnetic bead or a glass bead. A person skilled in the art is able to select any appropriate bead commonly used in this type of kits. Said solid particle is susceptible to be present as a suspension and to be sensitive to the application of an external physical force.

A solid particle of a kit according to the invention is responsive to an external physical force. Among physical forces, one can cite for example:
gravity, applicable via a centrifugation step,
electromagnetic force, applicable in the presence of a magnet, or
a shear force exerted by flow or filtration.

In a particular embodiment, a kit according to the invention comprises a magnetic bead covalently coupled to a functional unit F2. A magnetic bead coupled to a functional unit F2 is sensitive to a magnetic force.

An ultrabright luminescent particle exhibits a very elevated level of brightness. The brightness is defined as the product of the extinction coefficient and the fluorescence quantum yield. In a kit and a process according to the invention, an ultrabright luminescent particle (ULP) is any particle which is characterized by a brightness of at least 10⁷ M⁻¹ cm⁻¹, preferably at least 2×10⁷ M⁻¹ cm⁻¹, at least 5×10⁷ M⁻¹ cm⁻¹, at least 10×10⁷ M⁻¹ cm⁻¹, at least 40×10⁷ M⁻¹ cm⁻¹.

The brightness of a particle may also be defined by the ratio of dye weight and total particle weight. For an ultrabright particle in a kit or a process according to the invention, said ratio is preferably of at least 1 wt % dye loading, preferably at least 5 wt % dye loading, at least 10 wt % dye loading, at least 20 wt % dye loading, at least 30 wt % dye loading, at least 40 wt % dye loading or at least 50 wt % dye loading.

In a kit according to the invention, an ultrabright luminescent particle (ULP) may be chosen among any luminescent particle known by a person skilled in the art. Said particle is preferably chosen in the group consisting of: fluorescent, phosphorescent, chemiluminescent and bioluminescent particles. In a particular embodiment, said ultrabright luminescent particle is an ultrabright fluorescent particle.

In a particular embodiment of a kit according to the invention, a probe P2 comprises, or consists of, a nucleic acid fragment NA2 linked to an ultrabright fluorescent particle exhibiting a brightness of at least 10⁷ M⁻¹ cm⁻¹, preferably at least 2×10⁷ M⁻¹ cm⁻¹, at least 5×10⁷ M⁻¹ cm⁻¹, at least 10×10⁷ M⁻¹ cm⁻¹ or at least 40×10⁷ M⁻¹ cm⁻¹. More particularly, a kit according to the invention comprises a probe P2 comprising, or consisting of, a nucleic acid fragment NA2 linked to an ultrabright fluorescent polymeric nanoparticle exhibiting a brightness of at least 10⁷ M⁻¹ cm⁻¹, preferably at least 2×10⁷ M⁻¹ cm⁻¹, at least 5×10⁷ M⁻¹ cm⁻¹, at least 10×10⁷ M⁻¹ cm⁻¹ or at least 40×10⁷ M⁻¹ cm⁻¹.

In another particular embodiment, said ultrabright luminescent particle is an ultrabright luminescent nanoparticle. In even more particular embodiment, said ultrabright luminescent particle is an ultrabright luminescent polymeric nanoparticle.

Dye-loaded polymeric nanoparticles due to their high brightness and modularity are a promising sensitive alternative for the detection of nucleic acids in solution. Previously, to ensure high brightness while addressing the problem of aggregation-caused quenching of encapsulated dyes in these NPs, a concept of ionic dye insulation by bulky hydrophobic counterions has been proposed, yielding NPs ~100-fold brighter than semiconductor quantum dots (QDs) of similar size (Melnychuk & Klymchenko, 2018), (Reisch et al., 2014), (Reisch et al., 2017). Also, a charge-controlled nanoprecipitation of hydrophobic polymers bearing few charged groups was introduced to achieve controlled size of polymeric NPs (Reisch et al., 2015). The strategy based on charged amino acids for exposing azide groups at NPs surface allowed further modification with nucleic acids by click chemistry. The obtained DNA-NPs conjugates yielded ultrabright nanoprobes for amplified detection of DNA/RNA in solutions with picomolar limit of detection, and on surfaces with single-molecule sensitivity, compatible with smartphone-based sensing. Also, these DNA-functionalized NPs have already been validated for detection of microRNA in cell extracts, and for direct detection of RNA inside the cells by RNA-Fluorescence in situ hybridization protocols. Their application is explored for amplified enzyme-free detection of RNA in solution by a simple one-step protocol using total RNA cells extracts or serum medium.

By ultrabright dye-loaded polymeric nanoparticles is meant a particle loaded with fluorescent dyes and comprising at least 200, 500, 1000, 2000 or 10,000 fluorophore molecules, wherein one fluorophore molecule exhibit a brightness of at least 4·10⁴ M⁻¹ cm⁻¹. The ultrabright particle may in particular consist of polymers. Non-limiting examples of polymers are: polymethacrylates, aliphatic polyesters, polystyrenes, polyurethanes. Examples of polymethacrylates include, but are not limited to, poly(methyl methacrylate), poly(ethyl methacrylate), poly(butyl methacrylate). Examples of derivatives of polymethacrylate are poly(methyl methacrylate-co-methacrylic acid) (PMMA-MA), poly(methyl methacrylate-co-2-methacrylamidoethanesulfonic acid) (PMMA-SO3). Examples of aliphatic polyesters can be cited as, but are not limited to, polycaprolactone (PCL), poly(lactic acid) (PLA), poly(glycolic acid) (PGA), poly(lactide co-glycolide) (PLGA).

According to a more particular embodiment, a kit according to the invention comprises an ultrabright dye-loaded fluorescent nanoparticle, in particular such as described in EP 3 536 806.

In a particular embodiment of this first aspect, the present invention relates to a kit for the detection of a target nucleic acid in a sample, wherein said kit comprises at least:
i) a probe P1 comprising or consisting of a nucleic acid fragment NA1 linked to a functional unit F1, wherein said nucleic acid fragment NA1 comprises or consists of a nucleotide sequence complementary to the nucleotide sequence of a region T1 of said target nucleic acid molecule,
ii) a functional unit F2 bound to a solid surface,
iii) a probe P2 comprising or consisting of a nucleic acid fragment NA2 linked to an ultrabright dye-loaded luminescent particle, wherein the nucleic acid fragment NA2 comprises or consists of a nucleotide sequence complementary to that of a region T2 of the target nucleic acid.

In another particular embodiment of the invention, a kit of the invention comprises at least:
i) at least one probe P1 comprising, or consisting of, a nucleic acid fragment NA1 covalently linked to a functional unit F1, wherein NA1 comprises or consists of a nucleotide sequence complementary to the nucleotide sequence of a region T1 of said target nucleic acid,
ii) a functional unit F2 bound to a solid surface,
iii) at least one probe P2 comprising, or consisting of, a nucleic acid fragment NA2 linked to an ultrabright luminescent particle, and
iv) at least one probe P3 comprising or consisting of: a first part comprising, or consisting of, a nucleic acid fragment NA4 having a nucleotide sequence complementary to the nucleotide sequence of NA2 and a second part comprising, or consisting of, a nucleic acid fragment NA3 having a nucleotide sequence complementary to the nucleotide sequence of a region T3 of the target nucleic acid, wherein said first part and said second part are bound via a nucleotide linker.

A non-limiting illustration of the elements of a kit according to the invention, and of their respective interactions is shown in FIG. 1B.

In this embodiment of a kit according to the invention, probe P3, also designated as “post-it capture sequence” or “post-it probe”, comprises at least two parts:
a first part comprising, or consisting of, a nucleic acid fragment NA4 having a nucleotide sequence complementary to the nucleotide sequence of NA2 of probe P2, and
a second part comprising, or consisting of, a nucleic acid fragment NA3 having a nucleotide sequence complementary to the nucleotide sequence of a region T3 of the target nucleic acid, wherein T3 is distinct from T1,
   wherein said first part and said second part are bound via a nucleotide linker.

Said first part and second part of P3 are preferably oligonucleotides, and probe P3 is preferably a nucleic acid comprising, or consisting of, a sequence of at least 10 nucleotides, preferably comprising, or consisting of, a sequence of at least 10, 11, 12, 13, 14, 15, 16, 17, 18, 19, 20, 21, 22, 23, 24, 25, 30, 35, 40, 50, 80, 100, 120, or 150 nucleotides.

In a particular embodiment, the first part and second part of P3 are bound via a linker, preferably long of at least 5 nucleotides, preferably at least 10, 15, 20 or 40 nucleotides.

According to this particular embodiment, the nucleotide sequence of the nucleic acid fragment NA2 of probe P2, does not depend on the nucleotide sequence of the target nucleic acid. This embodiment presents the advantage of comprising a probe P2 comprising a nucleic acid fragment NA2 which nucleotide sequence is independent from the target to be detected.

According to another particular embodiment, a kit of the invention may comprise at least two probes P1, for example 2, 3, 4, 5 or n probes P1, wherein n is the total number of P1 probes in the kit. Each of said P1 probe is designated as P1-1, P1-2 or P1-n and comprises a nucleic acid fragment NA1, respectively designated as NA1-1, NA1-2 or NA1-n, which nucleotide sequence is respectively complementary to a nucleotide sequence T1-1, T1-2 or T1-n of the target nucleic acid. A kit according to the invention may comprise 1, 2, 3, 4, 5, 6, 7, 8, 9, 10 or more probes.

According to this embodiment, a kit of the invention comprises at least:
i) at least two probe P1 comprising, or consisting of, a nucleic acid fragment NA1 covalently linked to a functional unit F1, wherein NA1 comprises or consists of a nucleotide sequence complementary to the nucleotide sequence of a region T1 of said target nucleic acid,
ii) a functional unit F2 bound to a solid surface,
iii) at least one probe P2 comprising, or consisting of, a nucleic acid fragment NA2 linked to an ultrabright luminescent particle, and
iv) at least one probe P3 comprising or consisting of: a first part comprising, or consisting of, a nucleic acid fragment NA4 having a nucleotide sequence complementary to the nucleotide sequence of NA2 and a second part comprising, or consisting of, a nucleic acid fragment NA3 having a nucleotide sequence complementary to the nucleotide sequence of a region T3 of the target nucleic acid, wherein said first part and said second part are bound via a nucleotide linker.

According to another particular embodiment, a kit of the invention may comprise at least two, three or more probes P3. Each of said P3 probe differs from the other P3 probes in the nucleotide sequence of its first part and/or the nucleotide sequence of its second part.

For example, probe P3-NA4-1 comprises a nucleic acid fragment NA4-1 comprising or consisting of a nucleotide sequence complementary to a nucleotide sequence NA2-1 on a P2 probe.

For example, probe P3-NA3-1 comprises a nucleic acid fragment NA3-1 comprising or consisting of a nucleotide sequence complementary to a nucleotide sequence T3-1 on said target nucleic acid.

According to this embodiment, a kit of the invention may comprise 1, 2, 3 or n different P3 probes, wherein n designates the number of said P3 probe. A kit according to the invention may comprise 1, 2, 3, 4, 5, 6, 7, 8, 9, 10 or more P3 probes.

According to this embodiment, a kit of the invention comprises at least:
i) at least one probe P1 comprising, or consisting of, a nucleic acid fragment NA1 covalently linked to a functional unit F1, wherein NA1 comprises or consists of a nucleotide sequence complementary to the nucleotide sequence of a region T1 of said target nucleic acid,
ii) a functional unit F2 bound to a solid surface,
iii) a probe P2 comprising, or consisting of, a nucleic acid fragment NA2 linked to an ultrabright luminescent particle, and
iv) at least two probes P3 comprising or consisting of: a first part comprising, or consisting of, a nucleic acid fragment NA4 having a nucleotide sequence complementary to the nucleotide sequence of NA2 and a second part comprising, or consisting of, a nucleic acid fragment NA3 having a nucleotide sequence complementary to the nucleotide sequence of a region T3 of the target nucleic acid, wherein said first part and said second part are bound via a nucleotide linker.

The nucleotide sequences of multiple P1 probes and multiple P3 probes are respectively capable to bind to different regions of the target nucleic acid, wherein said target nucleic acid is a long size nucleic acid molecule, by “long size” it is intended a target nucleic acid of at least 35 nucleotides, at least 50, at least 100, at least 1000 nucleotides and above ″. The presence of said 1, 2, 3 or n probes allows for the detection of this long size nucleic acid molecule wherein it is intact or in a fragmented form in the sample.

According to another particular embodiment, a kit of the invention may comprise at least two probes P2, for example 2, 3, 4, 5 or n probes P2, wherein n is the total number of P2 probes in the kit.

Each of said P2 probe is designated as P2-1, P2-2 or P2-n and comprises a nucleic acid fragment NA2, respectively designated as NA2-1, NA2-2 or NA2-n, which nucleotide sequence is respectively complementary to the sequence of a nucleotide fragment NA4-1, NA4-2 or NA4-n of probe P3. A kit according to the invention may comprise 1, 2, 3, 4, 5, 6, 7, 8, 9, 10 or more probes P2.

According to this embodiment, each P2-probe may also differ from other P2 probes by the nature of the ultra-luminescent particle. Indeed, the use of P2-probes comprising different ULP may allow the multiplexing of a detection by using a kit according to the invention.

In the presence of two or more different P3 probes in a particular embodiment, a kit according to the invention may comprise one or more different P2 probes. On the contrary, in the presence of two or more different P2 probes in a particular embodiment, a kit according to the invention must comprise two or more different P2 probes, as each of said P3 probe should be adapted to hybridize to each of the P2 probe.

According to this embodiment, a kit of the invention comprises at least:
i) at least one probe P1 comprising, or consisting of, a nucleic acid fragment NA1 covalently linked to a functional unit F1, wherein NA1 comprises or consists of a nucleotide sequence complementary to the nucleotide sequence of a region T1 of said target nucleic acid,
ii) a functional unit F2 bound to a solid surface,
iii) at least one probe P2, preferably at least two probes P2, comprising, or consisting of, a nucleic acid fragment NA2 linked to an ultrabright luminescent particle, and
iv) at least one probe P3 comprising or consisting of: a nucleotide sequence complementary to the nucleotide sequence of NA2 and a nucleic acid fragment NA3 having a nucleotide sequence complementary to the nucleotide sequence of a region T3 of the target nucleic acid.

This is important for multiplexed detection of different regions of nucleic acids simultaneously. In this case, ULPs have preferably different spectral properties.

A kit according to the invention may also comprise any suitable element for performing the detection reaction, such as, for example, buffers.

The second aspect of the present invention concerns a process for the detection of a target nucleic acid molecule by a sandwich-test, in a sample prepared from a biological sample, said process comprising at least the steps of:
a) contacting, in conditions adapted to the hybridization of complementary nucleic acid sequences and to the formation of a non-covalent complex, at least:
   a probe P1 comprising, or consisting of a first nucleic acid fragment NA1 linked to a functional unit F1, wherein NA1 comprises or consists of a nucleotide sequence complementary to the nucleotide sequence of a region T1 of the target nucleic acid,
   a functional unit F2 bound to a solid surface, wherein said functional unit F2 is exhibiting a high affinity for F1 or is covalently bound to F1, and
   a probe P2 comprising, or consisting of, a nucleic acid fragment NA2 linked to an ultrabright luminescent particle exhibiting a brightness of at least 10⁷ M⁻¹ cm⁻¹, preferably at least 2×10⁷ M⁻¹ cm⁻¹, at least 5×10⁷ M⁻¹ cm⁻¹, at least 10×10⁷ M⁻¹ cm⁻¹ or at least 40×10⁷ M⁻¹ cm⁻¹,
b) applying an external physical force to the mixture of step a), in order to isolate the complex formed and
c) measuring the luminescence emission intensity associated with said non-covalent complex of nucleic acids.

The present invention therefore relates to a process belonging to the group of the sandwich-type tests, for the detection of a target nucleic acid in a sample. In a process according to the invention, the applied physical force is adapted to the nature of the solid surface of the kit and is easily chosen by a person skilled in the art of test.

In a particular embodiment, the present invention relates to a process for the in vitro detection of a target nucleic acid in a sample.

Among physical forces, one can cite for example:
gravity, applicable via a centrifugation step,
electromagnetic force, applicable in the presence of a magnet, or
a shear force exerted by flow or filtration.

In a particular embodiment, a process according to the invention comprises applying a centrifugation step, exerting an electromagnetic force or applying at least a filtration step. In a process according to the invention, by “applying a physical force” it is included applying one, two, three or more steps of said physical force.

In a particular embodiment, the present invention pertains to a process for the detection of a target nucleic acid molecule in a sample, wherein said functional unit F2 is bound to a solid surface, said solid surface being the surface of a solid particle. In a more particular embodiment, said solid particle is a magnetic bead or a glass bead.

In a process according to the invention, an ultrabright luminescent particle (ULP) is any luminescent particle characterized by a brightness of at least 10⁷ M⁻¹ cm⁻¹, preferably at least 2×10⁷ M⁻¹ cm⁻¹, at least 5×10⁷ M⁻¹ cm⁻¹, at least 10×10⁷ M⁻¹ cm⁻¹, at least 40×10⁷ M⁻¹ cm⁻¹, or which is characterized by a ratio of dye weight and total particle weight of at least 1 wt % dye loading, preferably at least 5 wt % dye loading, at least 10 wt % dye loading, at least 20 wt % dye loading, at least 30 wt % dye loading, at least 40 wt % dye loading or at least 50 wt % dye loading.

In a process according to the invention, an ultrabright luminescent particle (ULP) may be chosen among any luminescent particle known by a person skilled in the art. Said particle is preferably chosen in the group consisting of: fluorescent, phosphorescent, chemiluminescent and bioluminescent particles. In a particular embodiment, said ultrabright luminescent particle is an ultrabright fluorescent particle.

In a particular embodiment of a process according to the invention, a probe P2 comprises, or consists of, a nucleic acid fragment NA2 linked to an ultrabright fluorescent particle exhibiting a brightness of at least 10⁷ M⁻¹ cm⁻¹. More particularly, a process according to the invention comprises a probe P2 comprising, or consisting of, a nucleic acid fragment NA2 linked to an ultrabright fluorescent polymeric nanoparticle exhibiting a brightness of at least 10⁷ M⁻¹ cm⁻¹.

In another particular embodiment, said ultrabright luminescent particle is an ultrabright luminescent nanoparticle. In even more particular embodiment, said ultrabright luminescent particle is an ultrabright luminescent polymeric nanoparticle.

According to a more particular embodiment, a process according to the invention comprises an ultrabright dye-loaded fluorescent nanoparticle, in particular such as described in EP 3 536 806.

In a particular embodiment of a process according to the invention, a probe P2 comprises, or consists of, a nucleic acid fragment NA2 linked to an ultrabright fluorescent particle exhibiting a brightness of at least 10⁷ M⁻¹ cm⁻¹, wherein said nucleic acid fragment NA2 comprises or consists of a nucleotide sequence complementary to that of a region T2 of said target nucleic acid.

In another particular embodiment, the present invention pertains to a process for the detection of a target nucleic acid molecule in a sample, wherein step a) comprises contacting, at least,
at least one probe P1 comprising, or consisting of a nucleic acid fragment NA1 linked to a functional unit F1, wherein NA1 comprises or consists of a nucleotide sequence complementary to the nucleotide sequence of a region T1 of said target nucleic acid,
a functional unit F2 bound to a solid surface, wherein F2 is exhibiting a high affinity for F1 or is covalently bound to F1, and
a probe P2 comprising, or consisting of, a nucleic acid fragment NA2 linked to an ultrabright luminescent particle,
at least one probe P3, comprising, or consisting of a: a nucleotide sequence complementary to the nucleotide sequence of a region T3 of the target nucleic acid, T3 being distinct from T1, and a nucleotide sequence complementary to the nucleotide sequence of NA2, and
to form a mixture.

In another particular embodiment, the present invention pertains to a process for the detection of a target nucleic acid molecule in a sample, wherein step a) comprises contacting, at least:
at least two probes P1, wherein each of said probe P1 comprises or consists of: a nucleic acid fragment NA1, wherein each of said NA1 is designated as NA1-1 or NA1-2 and is respectively complementary to a nucleotide sequence T1-1 or T1-2 of the target nucleic acid.
a probe P2 comprising, or consisting of, a nucleic acid fragment NA2 linked to an ultrabright luminescent particle,
at least one probe P3, comprising, or consisting of:
a nucleotide sequence complementary to the nucleotide sequence of a region T3 of the target nucleic acid, T3 being distinct from T1, and
a nucleotide sequence complementary to the nucleotide sequence of NA2, and
to form a mixture.

In another particular embodiment, the present invention pertains to a process for the detection of a target nucleic acid molecule in a sample, wherein step a) comprises contacting, at least:
at least two probes P1, wherein each of said probe P1 comprises or consists of: a nucleic acid fragment NA1, wherein each of said NA1 is designated as NA1-1 or NA1-2 and is respectively complementary to a nucleotide sequence T1-1 or T1-2 of the target nucleic acid.
a probe P2 comprising, or consisting of, a nucleic acid fragment NA2 linked to an ultrabright luminescent particle,
at least two probe P3, wherein each of said probe P3 comprises or consists of:
a nucleic acid fragment NA3 comprising or consisting of a nucleotide sequence NA3-1 or NA3-2, respectively complementary of nucleotide sequences T3-1 and T3-2 of the target nucleic acid, T3-1 or T3-2 being different from T1, and
a nucleotide sequence complementary to the nucleotide sequence of NA2, and
to form a mixture.

In a third aspect, the present invention pertains to the use of a kit according to the invention for the detection of a target nucleic acid.

In said third aspect, the present invention also pertains to the use of a process according to the invention for the detection of a target nucleic acid.

A kit and a process according to the invention are suitable for the detection of target nucleic acids having variable size. Indeed, said kit and process according to the invention are suitable for the detection of target nucleic acids such as micro RNA or long-sized RNA, single or double stranded DNA of different size. Therefore, a kit and a process according to the invention can be used for the detection of target nucleic acids size is preferably comprised of at least 15 nucleotides.

In a fourth aspect, the present invention pertains to an in vitro diagnostic method for a pathology or a condition, wherein said method comprises the use of a kit or a process according to the invention for the detection and/or for the extraction of a target nucleic acid specific for said pathology or condition.

In particular, the present invention pertains to an in vitro diagnostic method for a pathology due to the presence of SARS Cov-2 virus, wherein said method comprises the use of a kit or a process according to the invention for the detection and/or for the extraction of a target nucleic acid from said virus.

The present invention is illustrated in more detail by the following figures and examples.

### FIGURES

FIGS. 1A and 1B represent particular embodiments of nucleic acid detection method according to the invention. FIG. 1A shows an ultrabright luminescent particle (ULP) functionalized with a nucleic acid fragment NA2 capable to directly hybridize with region T2 of the target nucleic acid. FIG. 1B shows an ultrabright luminescent particle (ULP) functionalized with a nucleic acid fragment NA2 capable to bind to region T2 of the target nucleic acid through an intermediate, or “post-it” probe P3.

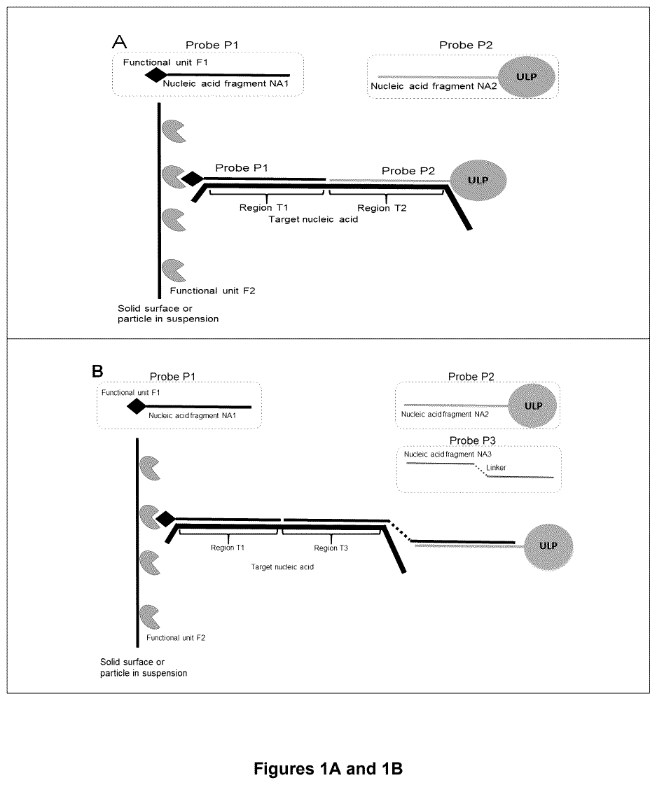

FIG. 2A represent a particular embodiment of the present invention in the presence of a target nucleic acid, wherein: the P1 probe is immobilized on a solid surface, it comprises a functional unit F1 and a nucleic acid fragment NA1, the P2 probe comprises a nucleic acid fragment NA2 and an ultrabright particle (NP). FIG. 2B shows the quantification of the number of particles in increasing concentrations of DNA target (SEQ ID N°6) (0, 0.001, 0.01, 0.1, 1 and 10 pM). PBS buffer with 0.01 g/L Tween 80 was systematically used for incubation and imaging.

FIG. 3A represents a particular embodiment of the present invention in the presence of a target nucleic acid, wherein: the P1 probe is to be immobilized on a solid particle in suspension, it comprises a functional unit F1 and a nucleic acid fragment NA1, the P2 probe comprises a nucleic acid fragment NA2 and an ultrabright particle (NP). FIG. 3B shows quantification of fluorescence intensity in increasing concentrations of DNA target (SEQ ID N°6) (0, 0.001, 0.01, 0.1, 1 and 10 pM). PBS buffer with 0.01 g/L Tween 80 was systematically used for incubation and detection.

FIGS. 4A and 4B represent detection of different length of target nucleic acid. FIG. 4A represents quantification of number of particles in detection of increased concentrations of 100 nucleotides RNA target (left bars for each concentration) or 1000 nucleotides RNA target (right bars for each concentration) (0, 1, 10, 100 and 1000 pM) by immobilized glass surface. FIG. 4B represents quantification of fluorescence intensity in detection of spiked increased concentrations of 100 nucleotides long RNA target (left bars for each concentration) or 1000 nucleotides long RNA target (right bars for each concentration) (0, 1, 10, 100 and 1000 pM) by magnetic beads sandwich detection in 2.5 μg of total RNA extract. RNA secure 1× buffer with 0.01 g/L Tween 80 was systematically used for incubation and detection. FIG. 4C represents 100 and 1000 nucleotides RNA targets with different location of target sequence complementary to DNA-NPs. FIG. 4D represents possible difficulties of direct hybridization between DNA-NPs and a target sequence located not in the target extremity.

FIG. 5A represents a particular embodiment of the present invention in the presence of a target nucleic acid, wherein: the P1 probe (Biotin capture) is immobilized on a solid particle in suspension, it comprises a functional unit F1 and a nucleic acid fragment NA1 that hybridizes with the target. The P2 probe comprises a nucleic acid fragment NA2 and an ultrabright particle (NP). The P3 probe (DNA post-it) hybridizes, on one part, to the target nucleic acid and, on the other part, to the nucleic acid fragment NA2 and an ultrabright particle (NP). The declining arrow on the right shows that after DNA-NP hybridization, there is no any steric hindrance at the extremity of the target. FIG. 5B represents the quantification of fluorescence intensity in detection of spiked increased concentrations of 48 nucleotides DNA target (SEQ ID N°6) (0, 0.01, 0.1, 1 and 10 pM) by magnetic beads sandwich detection in 2.5 μg of total RNA extract using either direct hybridization of DNA-NPs (white bars) or indirect hybridization through post-it probe (P3) (dark bars). PBS buffer with 0.01 g/L Tween 80 was systematically used for incubation and detection.

FIGS. 6A and 6B show the influence of NP characteristics in sandwich hybridization sensitivity. FIG. 6A shows the influence of NP size: Quantification of fluorescence intensity in detection of spiked increased concentrations of 48 nucleotides DNA target (SEQ ID N°6) (0, 0.1, 1 and 10 μM) by magnetic beads sandwich detection in 2.5 μg of total RNA extract using 20 nm or 50 nm core sized DNA-NPs in indirect hybridization through a P3 probe. FIG. 6B shows the influence of dye-loading: Quantification of fluorescence intensity in detection of spiked increased concentrations of 48 nucleotides DNA target (SEQ ID N°6) (0, 0.1, 1 and 10 μM) by magnetic beads sandwich detection in 2.5 μg of total RNA extract using 0.1, 1, 10 and 50 wt % dye-loaded DNA-NPs in indirect hybridization through post-it probe (P3 probe). RNA secure 1× buffer with 0.01 g/L Tween 80 was systematically used for incubation and detection.

FIGS. 7A and 7B show the detection method for long RNA target. FIG. 7A shows the workflow for sandwich detection of long RNA by indirect hybridization of DNA-NPs in suspended magnetic beads through post-it probe capture (P3). FIG. 7B shows the quantification of fluorescence intensity in detection of spiked increased concentrations of 100 nucleotides RNA target (SEQ ID N°23) or 1000 nucleotides RNA target (SEQ ID N°24) (0, 0.01, 0.1, 1 and 10 μM) by magnetic beads sandwich detection in 2.5 μg of total RNA extract using 50 nm core sized DNA-NPs in indirect hybridization through post-it probe (P3) and biotin capture with or without TEG motif in the 20 adenines linker. RNA secure 1× buffer with 0.01 g/L Tween 80 was systematically used for incubation and detection.

FIG. 8 shows the detection of SARS-COV-2 RNA in RNA extract from clinical samples. Quantification of fluorescence intensity in detection of infected sample CS7 (CT=18.75) diluted several times in negative sample CS1 (CT=37), while keeping constant total RNA (0.25 μg), using 50 nm core sized DNA-NPs and three biotin-captures (P1) and three post-it probe (P3) captures. RNA secure 1× buffer with 0.01 g/L Tween 80 was systematically used for incubation and detection.

FIGS. 9A to 9C represent the direct detection of SARS-COV-2 RNA from clinical samples directly without RNA extraction. FIGS. 9A and 9B represent the quantification of fluorescence intensity in detection of SARS-COV-2 RNA in clinical samples from two different sources (CS series and CH series), using 50 nm core sized DNA-NPs and three biotin-captures (P1) and three DNA post-its probes (P3). FIG. 9C represents several dilutions of a negative CS2 and positive CS6 sample in a buffer. RNA secure 1× buffer with 0.01 g/L Tween 80 was systematically used for incubation and detection.

FIG. 10 shows concentration dependent response of the assay on the glass surface for detection of MicroRNA targets: miR200a (left bar) and miR21 (right bar). Target concentration varies from 0 to 100 μM, the quantification of NPs varies from 0 to 25000. PBS buffer with 0.01 g/L Tween 80 was systematically used for incubation and detection.

### EXAMPLES

### Example 1: Short Nucleic Acid Detection

### 1.1. Material and Methods

Chemicals were purchased from either Sigma Aldrich, Alfa Aesar or Thermofisher Scientific. Polymer PMMA-AspN3-5% was synthesized as described previously (Melnychuk et al, 2020) (Melnychuk and Klymchenko, 2018). Rhodamine B octadecyl ester trakis (penta-fluorophenyl) borate (R18/F5) was synthesized by ion exchange and purified by column chromatography as described previously (Reish et al, 2014).

### Preparation of NPs

### NP-PEMA-MA-5%

50 μL of the polymer solution in acetonitrile (2 mg mL⁻¹ containing R18/F5 at 50 wt % relative to the polymer) were added quickly using a micropipette to 450 μl of 20 mM phosphate buffer, pH 7.4 50 mM NaCl at 21° C. under shaking (Thermomixer comfort, Eppendorf, 1100 rpm). While continuing mixing, 500 μL of 20 mM phosphate buffer, pH 7.4 50 mM NaCl were added. Then, the residues of acetonitrile were evaporated.

General Protocol for Nanoparticles Functionalization with DNA

Lyophilized single strand DNA sequences were purchased from Biomers or Eurogentec, dissolved in Milli-Q water, aliquoted and stored at −20° C. for further experiments. Aliquots of corresponding nucleic acid fragments linked to dibenzocyclooctine (DNA-DBCO) (SEQ ID N°1, SEQ ID N°8, SEQ ID N°14, SEQ ID N°19, SEQ ID N°20 or SEQ ID N°21) at a concentration of 20 μM in the reaction mixture, were added to 200 μl of corresponding nanoparticles. The reaction was mixed and kept overnight at 40° C. without shaking protected from light. Then the reaction was cooled down to room temperature. The mixture was diluted with 20 mM phosphate buffer to 4 mL and NPs were purified by centrifugation using centrifuge filters (Amicon, 0.5 mL, 100 kD, Sigma-Aldrich) on 1000 g at 20° C. for 5 min. The procedure of centrifugation was repeated 6 times to remove the non-reacted oligonucleotides using corresponding buffer. Upon each centrifugation, overflow was discarded and filtrate was resuspended in 4 mL 20 mM phosphate buffer This high level of purification is important to achieve high sensitivity of the method. The obtained functionalized DNA-NPs were kept in the dark at 4° C.

### Nanoparticle Characterization

Dynamic light scattering (DLS) measurements were performed on a Zetasizer Nano ZSP (Malvern Instruments S.A.). The Zetasizer software provided with standard cumulates and size distribution by volume analysis was used to characterize nanoparticles by DLS. For the data analysis, the following parameters were used: for the solvent (water)-temperature 25° C., refractive index RI 1.33, and viscosity 0.8872 cP. Nanoparticles were assumed to be all homogenous and spherical in shape. Absorption spectra were recorded on a Cary 5000 scan UV-visible spectrophotometer (Varian). Excitation, emission spectra and anisotropy were recorded on a FS5 Spectrofluoremeter (Edinburg Instruments). For standard recording of fluorescence spectra, the excitation wavelength was set 530 nm. The fluorescence spectra were corrected for detector response and lamp fluctuations.

### Transmission Electron Microscopy (TEM)

Carbon-coated copper-rhodium electron microscopy grids with a 300 mesh (Euromedex, France) were surface treated with a glow discharge in amylamine atmosphere (0.45 mbar, 5-5.3 mA, 25 s) in an Elmo glow discharge system (Cordouan Technologies, France). Then, 5 μL of the solution of NPs were deposited onto the grids and left for 2 min. The grids were then treated for 1 min with a 2% uranyl acetate solution for staining. They were observed with the Tecnai F20 electron microscope, equipped with a FEG operated at 200 keV. Areas covered with nanoparticles of interest were recorded at 29000× magnifications on a GATAN CCD 2K*2K “US10001” camera. Image analysis was performed using the Fiji software.

### Glass-Surface Nucleic Acid Detection

The LabTek chamber (Borosilicate cover glass, eight wells, ThermoFisher Scientific) was pre-treated with 1M of KOH for 30 minutes and washed 3 times with PBS followed by incubation with 100 μL of BSA-Biotin (Sigma-Aldrich) 0.5 mg mL⁻¹ in PBS for 20 min. Then, BSA-biotin solution was removed, and the chamber was washed 3 times with 500 μL of PBS. Next, chamber was incubated with 100 μL of neutravidin (ThermoFisher Scientific) solution (0.5 mg mL⁻¹ in PBS) for 15 min and washed 3 times with 500 μL of PBS. Then the chamber was incubated with 100 μL of 1 μM solution of P1 biotin-capture sequence (SEQ ID N°2) in PBS for 20 min and washed 3 times with PBS. Finally, 100 μL of detection solution was deposed with 200 μM of probe P2 and different concentration of synthetic target in PBS with 0.01 g/L of Tween80 and incubated for 1 hour at 40° C. in the dark. Before measurements the chamber was washed 3 times and covered with 200 μL of the same buffer.

Single-particle measurements were performed in the epi-fluorescence mode using Nikon Ti-E inverted microscope with a 20× objective (Apo TIRF, air, NA 1.4, Nikon). The excitation was provided by light emitting diodes (SpectraX, Lumencor) at 550 nm. The exposure time was set to 500 ms per image frame. The fluorescence signal was recorded with a Hamamatsu Orca Flash 4 camera.

Single-particle analysis was performed using the Fiji software. Particle locations were detected through a Fiji routine applied to a projection (maximum intensity) of all obtained frames per experiment. After the automatic background subtraction, the number of particles of interest with a diameter of 10 pixels were then measured. At least ten image sequences (1024 pixel×1024 pixel) per condition were analyzed.

### Magnetic Beads Nucleic Acid Detection

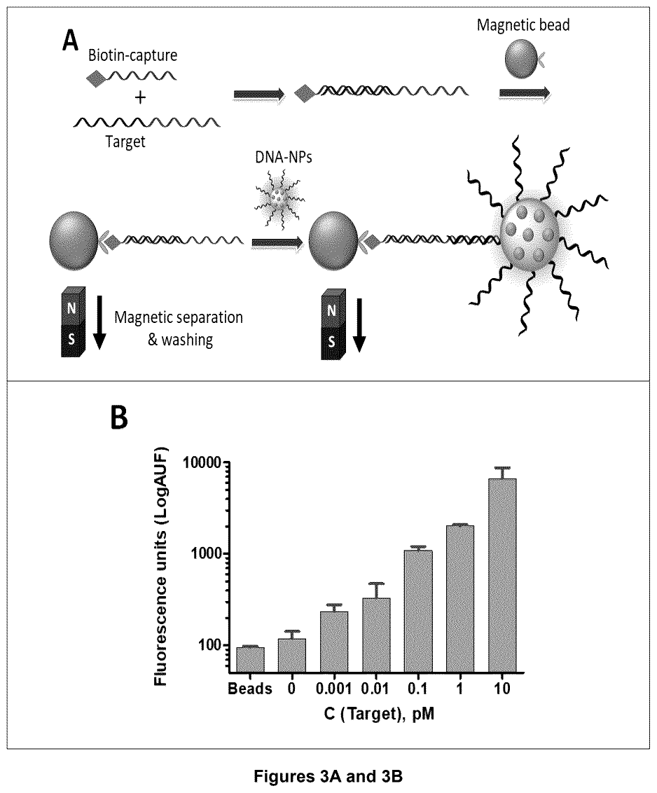

Synthetic target solution (SEQ ID N°6, in FIGS. 3, 5 and 6) at different concentrations on PBS with 0.01 g/L of Tween 80 were incubated with 50 nM P1 biotin-capture sequences and also 10 nM post-it probe (P3) capture sequence when indicated. Cell lysates and RNA extracts were diluted on RNA Secure (Sigma) with 0.01 g/L of Tween 80 and incubated with 20 nM of each biotin-capture sequences P1 and 10 nM of each post-it (P3) capture sequence P3 as indicated. Solution was heated at 95° C. for 30 seconds, cooled on ice for five minutes, and incubated at 40° C. during 20 min without agitation. Then 0.1 mg of streptavidin magnetic beads, previously washed twice with PBS, were added. The suspension was incubated at 40° C. during one hour with mild agitation. The complex composed by magnetic beads, target and capture sequences were separated from non-reacted nucleic acids by magnetic field. The beads were washed thrice with the same incubation buffer. 200 μM of nanoprobes (probe comprising SEQ ID N°19, SEQ ID N°20, or SEQ ID N°21) were added and incubated at 40° C. during one hour with mild agitation. The complex composed by magnetic beads, target, capture sequences and nanoprobes were separated from non-reacted nanoparticles by magnetic field. The beads were washed thrice with same incubation buffer, and complex was resuspended with 50 μL of the same buffer before transferring it to a 96-well black plate. Direct detection of nucleic acid was performed on beads surface by measuring intensity fluorescence in microplate (SPARK, TECAN).

Oligonucleotides and their respective nucleic acid sequences used in the examples are referenced in Table 1 and in the sequence listing annexed to the present patent application. N1, SPIKE and ORF1 respectively represent regions from SARS-COV-2 virus which were used as targets, and from which were designated nucleotide sequences of probes P1, P2 and P3. B, C and NS designate the respective non-coding sequences of probe P2, to be bound to NPs. NP designates an ultrabright nanoparticle. When using P3, NP are the same to detect all target sequences, P2 is not complementary to target, the P3 probe is different according to the target to be detected.

TABLE 1

| SEQ ID No | Short Name | Designation |
|---|---|---|
| 1 | N1 - NP | Probe P2, nucleotide sequence complementary to sequence |
| | | N1, to be functionalized with DBCO -TEG and linked to NP |
| 2 | N1 - Biotin | Probe P1, nucleotide sequence complementary to sequence |
| | | N1, to be linked to Biotin-TEG |
| 3 | N1 - SEQ B | Probe P3, nucleotide sequence complementary to sequence |
| | | N1 and nucleotide sequence complementary to sequence B |
| | | of probe P2 |
| 4 | N1 - SEQ C | Probe P3, nucleotide sequence complementary to sequence |
| | | N1 and nucleotide sequence complementary to sequence C |
| | | of probe P2 |
| 5 | N1 - NS | Probe P3, nucleotide sequence complementary to sequence |
| | | N1 and nucleotide sequence complementary to sequence NS |
| | | of probe P2 |
| 6 | N1 - small | Target nucleotide sequence of 48 nucleotides from N1 region |
| | target | of SARS-CoV-2 virus |
| 7 | N1 - long | Target nucleotide sequence of 1000 nucleotides from N1 |
| | target | region of SARS-CoV-2 virus |
| 8 | SPIKE - NP | Probe P2, nucleotide sequence complementary to sequence |
| | | SPIKE, to be functionalized to DBCO -TEG and linked to NP |
| 9 | SPIKE - | Probe P1, nucleotide sequence complementary to sequence |
| | Biotin | SPIKE, to be linked to Biotin-TEG |
| 10 | SPIKE - SEQ B | Probe P3, nucleotide sequence complementary to sequence |
| | | SPIKE and nucleotide sequence complementary to sequence |
| | | B of probe P2 |
| 11 | SPIKE - SEQ C | Probe P3, nucleotide sequence complementary to sequence |
| | | SPIKE and nucleotide sequence complementary to sequence |
| | | C of probe P2 |
| 12 | SPIKE - NS | Probe P3, nucleotide sequence complementary to sequence |
| | | SPIKE and nucleotide sequence complementary to sequence |
| | | NS of probe P2 |
| 13 | SPIKE - | Target nucleotide sequence from SPIKE region of |
| | small target | SARS-CoV-2 virus |
| 14 | ORF1 - NP | Probe P2, nucleotide sequence complementary to sequence |
| | | ORF1, to be functionalized with DBCO -TEG and linked to NP |
| 15 | ORF1 - Biotin | Probe P1, nucleotide sequence complementary to sequence |
| | | ORF1, to be linked to Biotin-TEG |
| 16 | ORF1 - SEQ B | Probe P3, nucleotide sequence complementary to sequence |
| | | ORF1 and nucleotide sequence complementary to sequence |
| | | B of probe P2 |
| 17 | ORF1 - SEQ C | Probe P3, nucleotide sequence complementary to sequence |
| | | ORF1 and nucleotide sequence complementary to sequence |
| | | C of probe P2 |
| 18 | ORF1 - NS | Probe P3, nucleotide sequence complementary to sequence |
| | | ORF1 and nucleotide sequence complementary to sequence |
| | | NS of probe P2 |
| 19 | SEQ B - NP | Probe P2, nucleotide sequence B, to be functionalized to |
| | | DBCO -TEG and linked to NP |
| 20 | SEQ C - NP | Probe P2, nucleotide sequence C, to be functionalized to |
| | | DBCO -TEG and linked to NP |
| 21 | NS - NP | Probe P2, nucleotide sequence NS, to be functionalized to |
| | | DBCO -TEG and linked to NP |
| 22 | ORF-small | Target nucleotide sequence from ORF1a region of |
| | target | SARS-CoV-2 virus |
| 23 | N1-163nt | Target RNA transcript nucleotide sequence of 163 |
| | target | nucleotides from N1 region of SARS-CoV-2 virus |
| 24 | N1-1362 nt | Target RNA transcript nucleotide sequence of 1362 |
| | target | nucleotides from N1 region of SARS-CoV-2 virus |

### 1.2. Results

This example discloses an embodiment of the invention wherein a single recognition event is combined with binding of ultrabright NPs to the solid support. Dye-loaded polymeric NPs were chosen because of their high brightness, due to large number of encapsulated dyes R18 with bulky counterion F5-TPB that minimizes self-quenching. To formulate NPs, polymer PEMA-AspN3 bearing azide and carboxylate functional group was used (Melnychuk et al., 2020). The hydrophobic PEMA block in this polymer provides good optical properties for the encapsulated dyes, while carboxylate ensures formation of small NPs and exposure of azide reactive group at NPs surface. NPs were formulated by nanoprecipitation of acetonitrile solution of PEMA-AspN3 into phosphate buffer (pH 7.4). R18/F5-TPB dye was loaded inside NPs at 50 wt % vs polymer (i.e. 33 wt % with respect to total particle mass) by premixing it with the polymer in acetonitrile before the nanoprecipitation, in line with previously develop methodology (Melnychuk et al., 2020). The obtained NPs were of 24.5 nm size with good polydispersity. Then, DNA (SEQ ID N°1) was grafted to the NPs surface using click reaction between DBCO group of DNA oligonucleotide and azides of polymeric NPs. The obtained NPs, purified by ultrafiltration, were 31.5 nm size according to DLS. The increase by ~7 nm corresponds to the double thickness of the DNA shell on NPs surface. The absorption and fluorescence spectra of the obtained DNA-NPs corresponded well to encapsulated R18/F5-TPB dye. The fluorescence quantum yield was 28%, in line with earlier works on analogous NPs. Given that dye loading is 50 wt % vs polymer and NPs size of 24.5 nm, the estimated number of dyes per NPs is ~1100. Thus, the brightness of NPs corresponds to ~1000 dyes with a high quantum yield.

To detect the target, two approaches based on a similar hybridization concept wherein the target DNA links NPs to a solid support were considered.

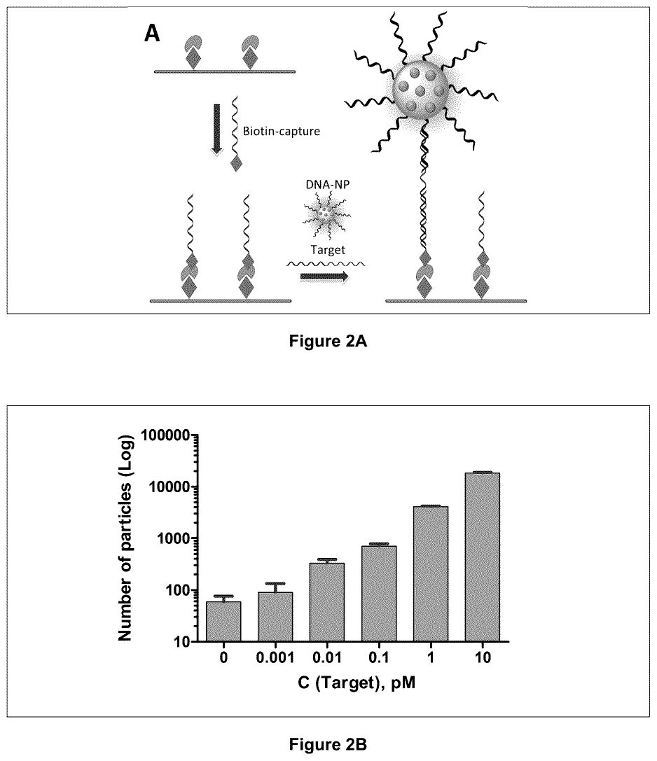

In a first approach, the glass surface and NPs surface are functionalized with two different capture DNA sequences (SEQ ID N°2, and SEQ ID N°1, respectively) (FIG. 2A). To functionalize the glass surface, an established approach based on adsorption of BSA-biotin, further binding of streptavidin and finally addition of corresponding DNA capture bearing biotin unit was used (FIG. 2A). In the presence of the target DNA/RNA, capture sequences would hybridize with the target, leading to immobilization of NPs on the glass surface. After a washing step, the latter can be further visualized by fluorescence microscopy. Given high brightness of these NPs, they could be directly visualized by a simple epi-fluorescence microscope using LED excitation light. As a target, a region N1 of SARS-COV-2 RNA was selected. Without the short target practically no NPs were detected at the NPs surface (Data not shown). Then, addition of the short target (SEQ ID N°6) lead to appearance of fluorescence dots at the surface and the signal increased clearly with the amount of the target (FIG. 2B). Thus, DNA-NPs did not bind non-specifically to the DNA-functionalized glass surface, whereas the target oligonucleotide led to immobilization of NPs on the glass surface. Importantly, the number of immobilized NPs correlated well with the concentration of the target oligonucleotides (FIG. 2B). The limit of detection in these measurements were in the 1-10 fM, which is remarkably low. The experiments with a non-coding DNA target gave low signal, indicating that the detection is sequence-specific (Data not shown). These experiments provide the proof of concept for nucleic acid detection by the target-driven DNA-NPs immobilization strategy.

Inspired by the obtained results on the glass surface, in order to simplify the detection scheme, where one would not need a fluorescence microscope for DNA/RNA detection, the glass surface was replaced with magnetic beads functionalized with streptavidin. Using magnetic beads has two important features. First, magnetic beads are well established for RNA extraction. Second, magnetic beads are easy to handle by magnetic steps, allowing efficient washing and further use in simple detection schemes, for example with help of a plate reader. Here, the target oligonucleotide (SEQ ID N°6) is hybridized with capture DNA-biotin (the same as in the case of glass-based approach) and further immobilized on the surface of magnetic bead (FIG. 3A). After washing the beads with help of magnetic stand, the resuspended beads were mixed with DNA-NPs, (the same as in the case of glass-based approach). In this case, the presence of the target is expected to immobilize DNA-NPs on the beads surface, while the excess of non-reacted DNA-NPs can be washed away using magnetic separation. As a result, magnetic beads hybridized through the target with fluorescent NPs are obtained. This complex could be resuspended and further detected by fluorescence plate reader, avoiding the use of expensive microscope. The result shows that the increases in the concentration of the target lead to increase in the fluorescence signal recorded by the plate reader (FIG. 3B). Without the target, the signal was very low, close to that for magnetic beads only. Thus, without the target, DNA-NPs do not interact with the beads, while the presence of the target triggers hybridization of DNA-NPs on the bead surface. The signal was already clearly observed at 1 fM, while estimated limit of detection was 0.3 fM. The obtained results are in line with those obtained with immobilization on glass, suggesting that target-triggered immobilization of DNA-NPs is a robust methodology for nucleic acid detection. However, in case of magnetic beads comparable or even better sensitivity was obtained, using simpler and much less expensive fluorescence instrument. To verify the sequence specificity of the oligonucleotide detection, the same beads-based experiments were performed, in the presence of the total RNA lysate from cells, which contains a large variety of RNA sequences. Importantly, similar dose-depended response was observed in the presence of total RNA extracted from cell lysate, so that numerous RNA sequences present in the lysate did not interfere with the hybridization processes. These results confirmed that the detection of the target nucleic acid is sequence specific. The obtained limit of detection was 1 fM, which was close to that without lysate.

### Example 2: Detection of Different Length of Target Nucleic Acid, Optionally in the Presence of a Third Probe

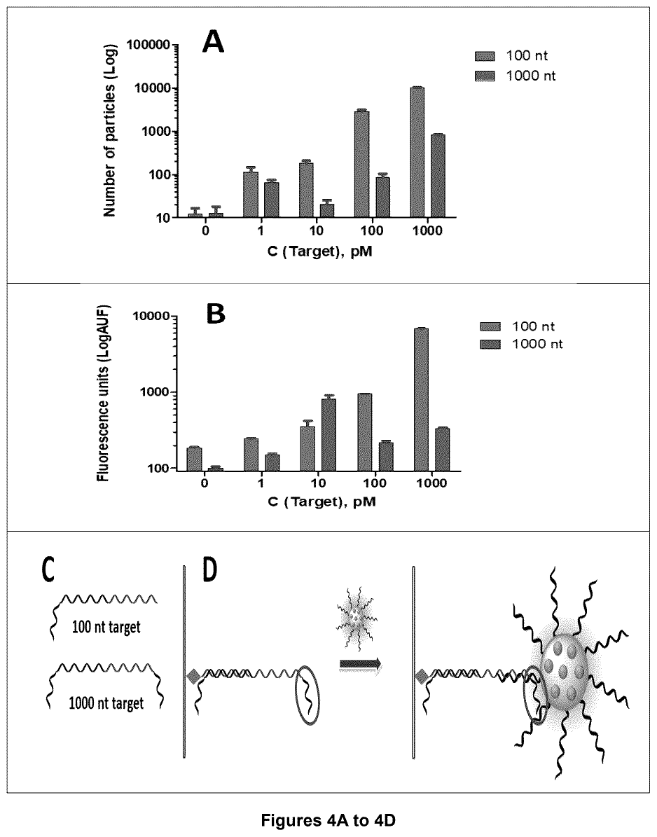

The viral RNA is a long and folded molecule presenting inaccessible regions, even if in biological or clinical samples the viral RNAs are generally present in form of fragments caused by decomposition of relatively unstable RNA molecules. Therefore, longer RNA sequences were studied. The first target (SEQ ID N°23) was 100 mer (100 nt), where sequence complementary to biotin-capture DNA of SEQ ID N°2 is located inside the sequence, while that complementary to the DNA-NPs of SEQ ID N°1 was at its the extremity. The second target (SEQ ID N°24) was 1000 nt, where both target sequences were located inside the total sequence. They were tested using both glass surface and magnetic bead-based approaches. Results show that 100 nt RNA target could be readily detected by both methods (FIGS. 4A and 4B), showing clear dose-dependent in the particle number (glass surface method) or fluorescence intensity (magnetic beads method). However, it can be noted that the particle number and the fluorescence intensity dropped in both cases compared to 48 nt. Indeed, values obtained when detecting 48 nucleotides targets are 10 to 100 times higher than when detecting the same concentration of 100 nucleotides long nucleic acid. Given the target length is not so different, this significant decrease in the performance of these two techniques is probably linked to the fact that sequence complementary to the biotin-capture is located inside the total sequence of 100 nt, posing steric issues for the interaction with streptavidin of the magnetic bead. On the other hand, the 1000 nt RNA target did not show a clear response, even though some increase in the signal was observed for samples with target compared to the control (FIGS. 4A and 4B). Therefore, combination of long target with presence of internal sequence complementary to DNA-NPs decreases performance our hybridization approach, independently of the nature of the immobilization surface (glass or bead). It can be speculated that it is sterically difficult for DNA-NPs to hybridize with this type of target because it would require unfavorable and sterically hindered turn in the RNA strand. Therefore, in the next steps a series of optimizations was made for both hybridization mechanism and particles itself, while focusing on a simpler method based on magnetic beads.

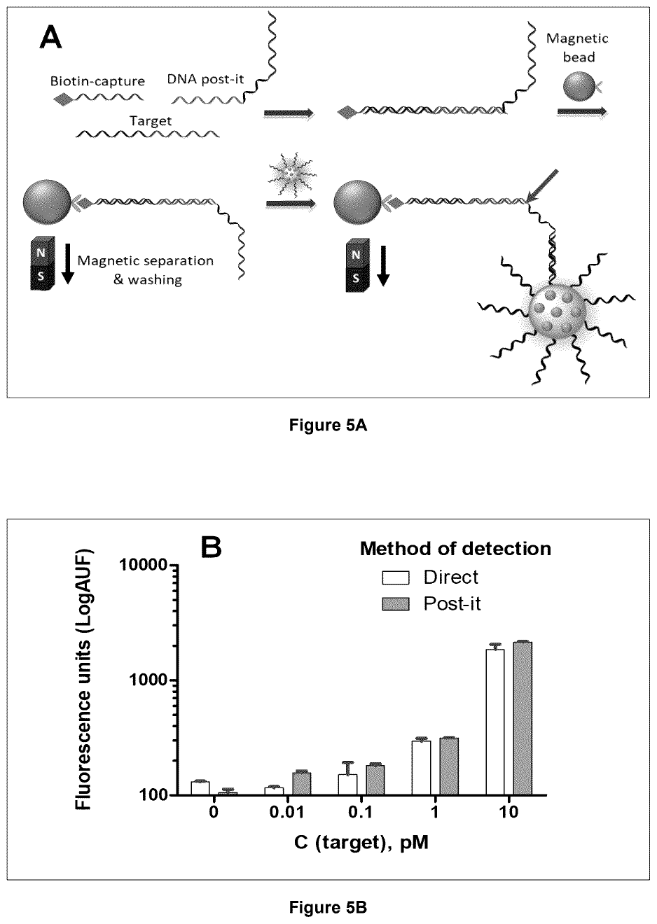

To improve hybridization of NPs with the target sequence located inside the total sequence, the inventors designed a third probe P3, designated as a “post-it” sequence, with one end complementary to the target and another end complementary to DNA-NPs, connected by a A20 linker (FIG. 5A). In this case, the steric problem with DNA-NPs is completely solved. To validate the method, this approach was tested on a target nucleotide sequence of 48 nucleotides from N1 region of SARS-COV-2 virus (SEQ ID N°6) and found that the signal showed a clear dose dependent response as shown in FIG. 5B. The application of this method to long RNA sequences is shown below.

### Example 3: Influence of NP Characteristics in Sandwich Hybridization Sensitivity

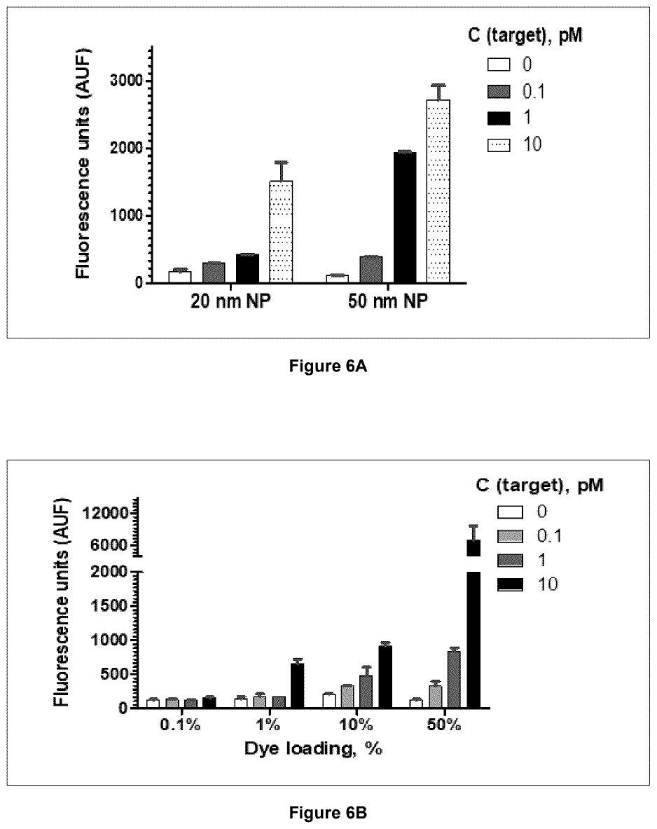

To increase the signal obtained within this assay, the number of encapsulated dyes per particle was varied. At first, the size of NPs was increased. This was achieved by nanoprecipitation of the same polymer at higher concentration of salt. In the presence of 50 mM NaCl in phosphate buffer, NPs of 52.8 nm polymer core according to DLS were obtained. TEM images showed particle size of 35±8 nm, which corresponds to ~3200 dyes per particle. The absorption and fluorescence spectra of these NPs were similar to those of parent smaller analogues. Taking into account that these NPs exhibited good fluorescence quantum yield (41%), they are expected to be ~3-times brighter that the original formulation. Their fluorescence brightness corresponds to 3200×0.41×125000=1.6×108 M⁻¹ cm⁻¹. These brighter NPs were tested using the hybridization method with a post-it (Probe P3) DNA and significant improvement in the signal intensity for all tested 48 nt target concentrations was found (FIG. 6A). Thus, larger particles indeed provide stronger signal. Next, using the larger NPs, the importance of the dye loading was verified (FIG. 6B). Using the same DNA post-it method, it was observed that NPs with low dye loading 0.1 and 1 wt % showed poor signal for target concentrations below 10 μM. By contrast, at 10 and especially 50 wt % loading, the signal could be clearly observed at all tested 48 nt target concentrations, including 0.1 μM. It can be can concluded that for the magnetic bead method high brightness of DNA-NPs is of outmost importance: ~64 dyes per NP (1 wt % dye loading) is not sufficient for good performance of the assay, while good results are observed for ~640 dyes per NP (10 wt % loading). This is probably because at low dye loading, the scattering signal from magnetic beads is comparable to the signal from bound NPs. However, for ultrabright NPs, only few hybridized DNA-NPs is sufficient to detect the signal, so that very low concentrations of the target are needed. Therefore, in the next experiments, focus was made on large NPs at 50 wt % dye loading.

### Example 4: Optimized Conditions

The importance of the linker between the biotin and the capture sequence was determined by testing A10 and A20 linkers (Haider et al., 2016). Longer linker showed advantages in terms of lower background noise for control without the target, which improved sensitivity of the method.

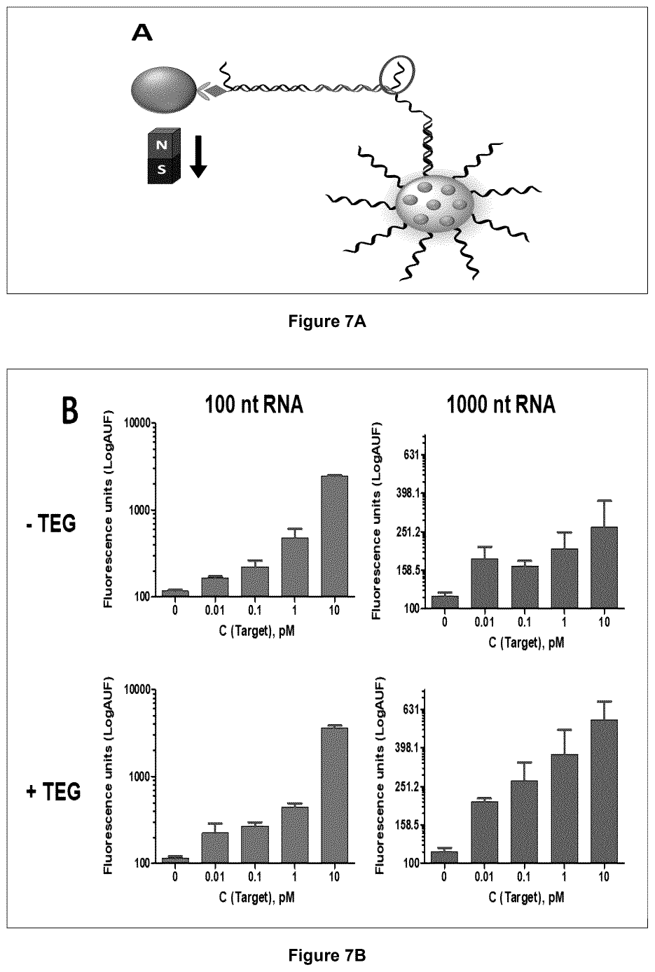

Finally, the optimized conditions contained the following improvements: (1) increase in the particle size to 50 nm, while keeping 50 wt % dye loading; (2) DNA post-it (probe P3); (3) A20 linker for the biotin-capture DNA. Using this optimized methodology (FIG. 7A), 100 and 1000 nt RNA targets were tested, which were spiked into RNA lysate.

In addition, the importance of a tetraethylene glycol (TEG) linker between the oligonucleotide and biotin, which was reported to improve the biotin capture by the beads was verified. In case of 100 nt target, an excellent response of the optimized assay was observed independently of the presence of TEG linker (FIG. 7B). The response was better compared to the original method: the detectable signal was clearly seen for 0.01 pM target vs 1 pM for the original method. Most importantly, the new method was able to clearly detect 0.01 pM of 1000 nt target, while the dose-response was better visible for the biotin-capture with TEG. These results provided a robust proof of concept for viral RNA detection by a new method combining ultrabright DNA-NPs with magnetic beads capture.

### Example 5: Test of Clinical Samples

Clinical samples were then tested using the optimized RNA sensing assay. The SARS-COV-2 RNA is long and contains many potential target sequences that could be detected. Three pairs of target sequences were identified in different parts of the viral genome. These SARS-COV-2 sequences are respectively: N1 (SEQ ID N°6, SEQ ID N°7, SEQ ID N°23, SEQ ID N°24), spike (SEQ ID N°13) and ORF1a (SEQ ID N°22). Three biotin captures (SEQ ID N°2, SEQ ID N°9, SEQ ID N°15) and three DNA post-it probes (SEQ ID N°3, SEQ ID N°4, SEQ ID N°5, SEQ ID N°10, SEQ ID N°11, SEQ ID N°12, SEQ ID N°16, SEQ ID N°17, SEQ ID N°18) were designed (including one mentioned above). It was expected that the use of 3 pairs of primers would significantly increase the probability of viral RNA capture as well as hybridization of the DNA-NPs.

The clinical samples used were collected from Department of Microbiology of University Hospital Center of Strasbourg (CHU) or bought from the company CliniSciences. In total, ten nasopharyngeal swab samples were from patients with qRT-PCR-positive SARS-COV-2 and ten nasopharyngeal swab samples were from qRT-PCR-negative patients. All samples arrived in inactivating transport medium. RNA extracts were prepared from all twenty samples using minispin column kit.

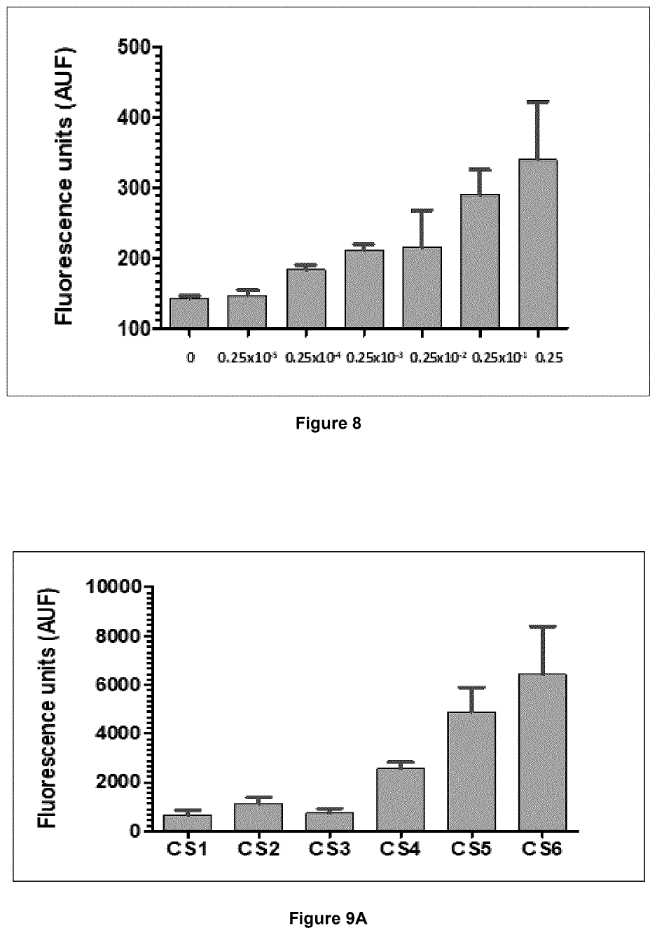

First, the response of the optimized assay was tested for detection of SARS-COV-2 RNA in RNA extracts from patients. The concentration of the target was varied by diluting RNA extract from SARS-COV-2 positive sample into that of SARS-COV-2 negative sample, while keeping constant the total RNA concentration. It is clearly observed that the SARS-COV-2 positive RNA extract showed much stronger fluorescence signal compared to the negative one (FIG. 8). The dilution of the positive sample let to gradual decrease in the signal, indicating that the response is dose-dependent. Remarkably, it was possible to see the significant response even after 10,000-fold dilution of the positive sample (CT=18.75), indicating that theoretically, this approach could detect SARS-COV-2 RNA in the RNA extract with sensitivity equivalent to ~30 PCR cycles.

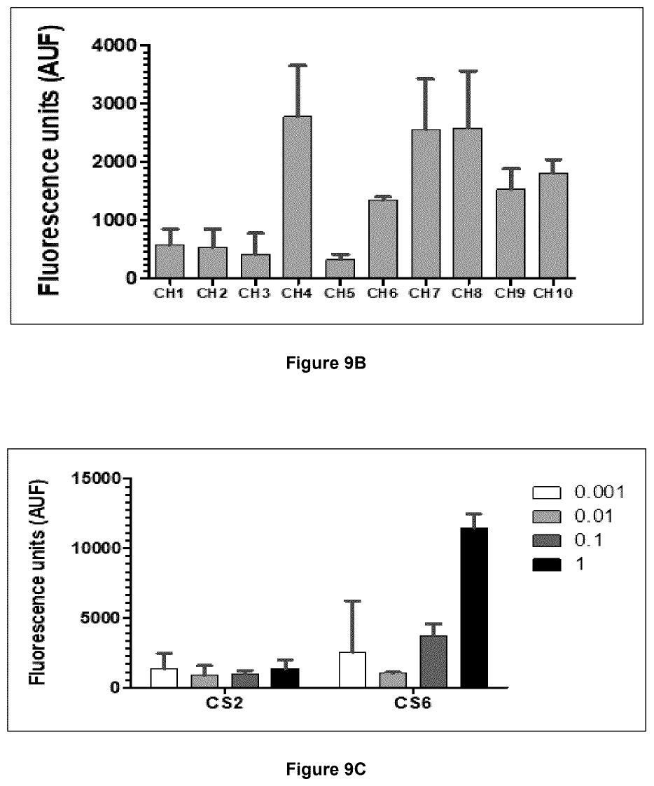

Ultimately, it was attempted to detect SARS-COV-2 RNA directly from clinical samples without RNA extraction. The hypothesis is that magnetic beads, which are commonly used for RNA extract, could effectively capture viral RNA with help of biotin-capture DNAs. Moreover, protocol includes magnetic separation with washing before application of DNA-NPs, which ensure that the buffer used in the original clinical sample would not harm DNA-NPs. Two series of clinical samples from two different sources were used: commercial source (CliniSciences, CS series) and from the university hospital CHU (CH series). Direct detection of these samples showed that for CS series all three tested SARS-COV-2 positive samples showed higher signal compared to the three negative ones (FIG. 9A). For CH series, the response of five tested positive samples showed systematically higher signal compared to four out of five controls (FIG. 9B), so that only one out of 10 tested CH samples showed false positive result. Then, dilutions of the samples were prepared in a buffer and found that in the SARS-COV-2 the signal decreased with the dilution, which was not the case of the control (FIG. 9C). These results confirm the capacity of DNA-NPs based assay to detect SARS-COV-2 directly in the clinical sample without dedicated RNA extraction step.

As a conclusion, a kit and a process according to the invention therefore represent highly sensitive nucleic acid sensing methodology. Target-driven immobilization of DNA-NPs on a solid surface allows detection of RNA/DNA target. A kit and a process of the invention could enable simple and automated high-throughput nucleic acid, and particularly RNA, detection in biological samples. Brightness of NPs plays an essential role in obtaining good fluorescence signal even for low RNA concentration, allowing LOD in the sub-fM range.

For the detection of long nucleic acid, probably because of slower diffusion kinetics and RNA folding that can block target sequence accessibility from inside the total sequence, the presence of a third probe designated to be an intermediate between the long target nucleic acid and ultrabright particle, and a linker of sufficient length for the biotin-capture DNA are important. The optimized assay allowed detection of 1000 nt RNA fragment of SAR-COV-2 with fM sensitivity. Moreover, it showed capacity to detect SARS-COV-2 in clinical samples with sensitivity equivalent to 19-30 RT-PCR cycles.

Ultimately, thanks to combination of DNA-NPs with magnetic beads, the method was able to detect SARS-COV-2 directly in the clinical samples without dedicated RNA extraction step. This work presents a new methodology for simple and rapid RNA detection, where ultrabright DNA-NPs provide ultimate sensitivity, while the use of magnetic beads allows to bypass RNA extraction and enables the use of simple detection mode by a plate reader.

As a conclusion, the inventors designed a highly sensitive RNA sensing methodology, which is based on ultrabright DNA-functionalized dye-loaded polymeric NPs, magnetic beads and corresponding primers. Target-driven immobilization of DNA-NPs on solid support allows detection of RNA/DNA target on the glass surface by fluorescence microscopy and directly on magnetic beads by a fluorescence plate reader. The latter method is of particular interest because it could enable simple and automated high-throughput RNA detection in biological samples. Brightness of NPs plays an essential role in obtaining good fluorescence signal even for low RNA concentration, allowing LOD in the fM and even in sub-fM range. Moreover, detection of long RNA was found challenging, probably because of slower diffusion kinetics and RNA folding that can block target sequence accessibility from inside the total sequence. Therefore, a DNA post-it (Probe P3) capture sequence was implemented as an intermediate between the long RNA and our DNA-NPs and ensure sufficient linker length for the biotin-capture DNA. The optimized assay allowed detection of 1000 nt RNA fragment of SARS-COV-2 with fM sensitivity. Moreover, it showed capacity to detect SARS-COV-2 in clinical samples with sensitivity equivalent to 19-30 RT-PCR cycles. Ultimately, thanks to combination of DNA-NPs with magnetic beads, the method was able to detect SARS-COV-2 directly in the clinical samples without dedicated RNA extraction step. This work presents a new methodology for simple and rapid RNA detection, where ultrabright DNA-NPs provide ultimate sensitivity, while the use of magnetic beads allows to bypass RNA extraction and enables the use of simple detection mode by a plate reader. The method could be readily extendable for other types of RNA, which enable rapid molecular diagnostics of various diseases, especially viral infections and cancers.

### Example 6: Detection of Micro-RNA Targets

For the detection of microRNA (miRNA) targets, which are 20-22 bases long, the inventors designed a simple system based on hybridization between complementary sequences. The system comprises of a capturing sequence (10-12 bases) on the glass surface that is complementary to one side of the miRNA target and NPs (ULP) coated in nucleic acids (10-12 bases) complementary to the other side of the miRNA target acting as detection probes. Locked Nucleic Acids (LNA) were incorporated in the capturing sequence at the glass surface in order to enhance the stability of the short duplex. LNAs were not integrated in the complementary oligonucleotides at the surface of NP, because the formed duplexes were stable enough, probably due to phenomenon of cooperativity of neighboring DNA grafted to NPs.

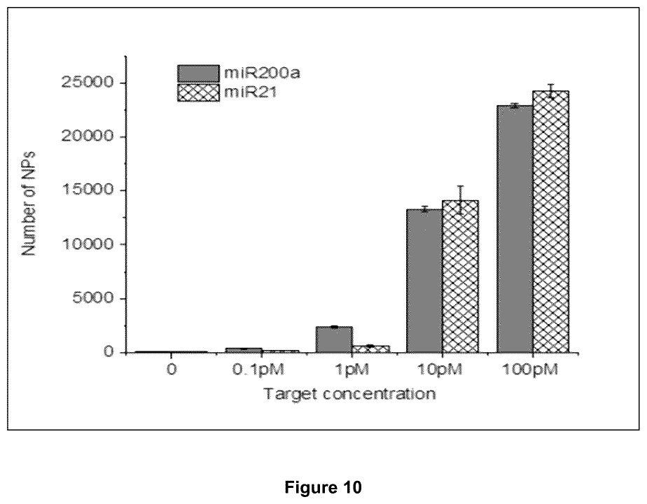

Oligonucleotides and their respective nucleic acid sequences used in the example (RNA or DNA, DNA target is shown in FIG. 10), biotin capture and recognition sequence (DBCO miR): are referenced in Table 2 and in the sequence listing annexed to the present patent application.

In the present patent application, the symbol “T” represents thymine in DNA, the symbol “U” represents uracil in RNA. As required by the ST26 standard, in the annexed sequence listing the symbol “T” will be construed as thymine in DNA and uracil in RNA.

In the present patent application, in SEQ ID N°27 and SEQ ID N°31 of Table 2, Locked Nucleic Acids are represented as bold and underlined characters. As required by the ST26 standard, in the annexed sequence listing, modified nucleotides such as Locked Nucleic Acids are represented in the sequence as the corresponding unmodified nucleotides.

TABLE 2

| | | Nature | | |
|---|---|---|---|---|
| SEQ | Short | and | | |
| ID No | Name | length | Designation | Sequence |
| 25 | miR-21 | RNA | Target | UAGCUUAUCAGACUGAUGUUGA |
| | RNA | 22 | nucleotide | |
| | | | sequence, RNA | |
| 26 | miR-21 | DNA | Target | TAGCTTATCAGACTGATGTTGA |
| | DNA | 22 | nucleotide | |
| | | | sequence, DNA | |
| 27 | miR21 | DNA | NA1 of probe P1, | AAAAAAAAAAAAAAAAAAAATCAACATCAGT |
| | Biotin | 31 | complementary | |
| | | | to sequence | |
| | | | miR21, linked to | |
| | | | Biotin-TEG | |
| 28 | miR21-NP | DNA | NA2 of probe P2, | CTGATAAGCTAAAAAAAAAAA |
| | | | complementary | |
| | | | to sequence | |
| | | | miR21, linked to | |
| | | | DBCO | |
| 29 | miR-200a | RNA | Target | CAUCUUACCGGACAGUGCUGGA |
| | RNA | | nucleotide | |
| | | | sequence, RNA | |
| 30 | miR-200a | DNA | Target | CATCTTACCGGACAGTGCTGGA |
| | DNA | | nucleotide | |
| | | | sequence, DNA | |
| 31 | Biotin- | DNA | NA1 of probe P1, | AAAAAAAAAATCCAGCACTGT |
| | miR200a | | complementary | |
| | | | to sequence | |
| | | | miR200a, linked | |
| | | | to Biotin-TEG | |
| 32 | miR200a- | DNA | NA2 of probe P2, | CCGGTAAGATGAAAAAAAAAA |
| | DBCO | | complementary | |
| | | | to sequence | |
| | | | miR200a, linked | |
| | | | to DBCO | |

The target oligonucleotide is hybridized with capture DNA-biotin and further immobilized on the glass surface. After washing the glass surface the particles were observed by a microscope.

When target is present, it hybridizes with the surface capture sequence on one side and with the complementary DNA of NP on the other side, immobilizing in this way the NP and allowing detection by microscope. In the absence of target the NPs remain in solution and are consequently washed out.

The LOD (Limit Of Detection) was calculated for the targets shown in FIG. 10, and is 4.5 fM for miR200a and 2 fM for miR21 (DNA version). The results for RNA version of microRNA were very similar.

These results show that relatively short oligonucleotides attached to NPs (10-12 bases) can be sufficient for microRNA detection in a sandwich assay.

### REFERENCES

Cai H. et al. Optofluidic analysis system for amplification-free, direct detection of Ebola infection. Sci Rep 5, 14494 (2015).
Egloff S. et al. Enzyme-free amplified detection of cellular microRNA by light-harvesting fluorescent nanoparticle probes. Biosens Bioelectron 179, 113084 (2021)
Haider M. et al. A Double-Hybridization Approach for the Transcription- and Amplification-Free Detection of Specific mRNA on a Microarray, Microarrays, March 5(1):5, 2016.
Lim S. H. et al Quantitative Analysis of Nucleic Acid Hybridization on Magnetic Particles and Quantum Dot-Based Probes. Sensors 9, 5590-5599 (2009).
Melnychuk & Klymchenko, DNA-Functionalized Dye-Loaded Polymeric Nanoparticles: Ultrabright FRET Platform for Amplified Detection of Nucleic Acids. J. Am. Chem. Soc. 140, 10856-10865 (2018).
Melnychuk et al. Light-Harvesting Nanoparticle Probes for FRET-Based Detection of Oligonucleotides with Single-Molecule Sensitivity. Angewandte Chemie International Edition 59, 6811-6818 (2020).
Ngo H. T. et al. Direct Detection of Unamplified Pathogen RNA in Blood Lysate using an Integrated Lab-in-a-Stick Device and Ultrabright SERS Nanorattles. Sci Rep 8, 4075 (2018).
Reisch A. et al. Collective fluorescence switching of counterion-assembled dyes in polymer nanoparticles. Nat Commun 5, 4089 (2014).
Reisch A. et al. Tailoring Fluorescence Brightness and Switching of Nanoparticles through Dye Organization in the Polymer Matrix. ACS Appl. Mater. Interfaces 9, 43030-43042 (2017).
Reisch A. et al. Charge-Controlled Nanoprecipitation as a Modular Approach to Ultrasmall Polymer Nanocarriers: Making Bright and Stable Nanoparticles. ACS Nano 9, 5104-5116 (2015).
Severi C. et al. Smartphone-assisted detection of nucleic acids by light-harvesting FRET-based nanoprobe. Biosensors and Bioelectronics 168, 112515 (2020)
Zheng Z. et al. Sensitive and quantitative measurement of gene expression directly from a small amount of whole blood. Clin Chem 52, 1294-1302 (2006).

1-15. (canceled)

16. A kit for the detection of a target nucleic acid in a sample, comprising:
i) a probe P1 comprising, or consisting of, a nucleic acid fragment NA1 linked to a functional unit F1, wherein NA1 comprises or consists of a nucleotide sequence complementary to the nucleotide sequence of a region T1 of the target nucleic acid,
ii) a functional unit F2 bound to a solid surface, wherein F2 is exhibiting a high affinity for F1 or is covalently bound to F1,
iii) a probe P2 comprising, or consisting of, a nucleic acid fragment NA2 linked to an ultrabright luminescent particle exhibiting a brightness of at least 10⁷ M⁻¹ cm⁻¹, preferably at least 2×10⁷ M⁻¹ cm⁻¹, more preferably at least 5×10⁷ M⁻¹ cm⁻¹, 10×10⁷ M⁻¹ cm⁻¹ or at least 40×10⁷ M⁻¹ cm⁻¹.

17. The kit according to claim 16, wherein said solid surface is chosen among an immobilized surface and the surface of a solid particle, preferably a magnetic or a glass bead.

18. The kit according to claim 16, wherein said ultrabright luminescent particle is an ultrabright dye-loaded fluorescent polymeric nanoparticle.

19. The kit according to claim 16, wherein said nucleic acid fragment NA2 comprises or consists of a nucleotide sequence complementary to that of a region T2 of said target nucleic acid.

20. The kit according to claim 16, further comprising at least one probe P3 comprising or consisting of: a first part comprising, or consisting of a nucleic acid fragment NA3 having a nucleotide sequence complementary to the nucleotide sequence of a region T3 of the target nucleic acid, T3 being distinct from T1, and a second part comprising, or consisting of, a nucleic acid fragment NA4 having a nucleotide sequence complementary to the nucleotide sequence of NA2, wherein said first part and said second part are bound via a nucleotide linker.

21. The kit according to claim 20, comprising at least two probes P1, each of said probe P1 comprising or consisting of:
a nucleic acid fragment NA1, designated respectively at least as NA1-1 or NA1-2 and respectively complementary to a nucleotide sequence T1-1 or T1-2 of the target nucleic acid.

22. The kit according to claim 20, comprising:
i) at least two probes P3, comprising or consisting of: a first part comprising, or consisting of, a nucleic acid fragment having a nucleotide sequence complementary to the nucleotide sequence of NA2 and a second part comprising, or consisting of, a nucleic acid fragment NA3 having a nucleotide sequence complementary to the nucleotide sequence of a region T3 of the target nucleic acid, wherein said first part and said second part are bound via a nucleotide linker, and
ii) at least one probe P2 comprising, or consisting of, a nucleic acid fragment NA2 linked to an ultrabright luminescent particle.

23. A process for the detection of a target nucleic acid molecule in a sample, wherein said process comprises at least the steps of:
a) contacting, in conditions adapted to the hybridization of complementary nucleic acid sequences and to the formation of a non-covalent complex of nucleic acids, at least:
a probe P1 comprising, or consisting of, a nucleic acid fragment NA1 linked to a functional unit F1, wherein NA comprises or consists of a nucleotide sequence complementary to the nucleotide sequence of a region T1 of said target nucleic acid,
a functional unit F2 bound to a solid surface, wherein F2 is exhibiting a high affinity for F1, or is covalently bound to F1, and
a probe P2 comprising, or consisting of, a nucleic acid fragment NA2 linked to an ultrabright luminescent particle, said particle exhibiting a brightness of at least 10⁷ M⁻¹ cm⁻¹, to form a mixture,
b) applying an external physical force to the mixture of step a), in order to isolate said non-covalent complex of nucleic acids, and
c) measuring the luminescence emission intensity associated with said non-covalent complex of nucleic acids.

24. The process according to claim 23, wherein said solid surface is the surface of a solid particle, preferably a magnetic or a glass bead.

25. The process according to claim 23, wherein said ultrabright luminescent particle is an ultrabright dye-loaded fluorescent polymeric nanoparticle.

26. The process according to claim 23, wherein said nucleic acid fragment NA2 comprises or consists of a nucleotide sequence complementary to that of a region T2 of said target nucleic acid.

27. The process according to claim 23, wherein step a) comprises contacting, at least:
at least probe P1 comprising, or consisting of, a nucleic acid fragment NA1 linked to a functional unit F1, wherein NA1 comprises or consists of a nucleotide sequence complementary to the nucleotide sequence of a region T1 of said target nucleic acid,
a functional unit F2 bound to a solid surface, wherein F2 is exhibiting a high affinity for F1, or is covalently bound to F1, and
at least one probe P2 comprising, or consisting of, a nucleic acid fragment NA2 covalently linked to an ultrabright fluorescent particle, wherein said nucleic acid fragment NA2 comprises or consists of a nucleotide sequence complementary to a probe P3, and
at least one probe P3 comprising or consisting of: a first part comprising, or consisting of, a nucleic acid fragment NA4 having a nucleotide sequence complementary to the nucleotide sequence of NA2 and a second part comprising, or consisting of, a nucleic acid fragment NA3 having a nucleotide sequence complementary to the nucleotide sequence of a region T3 of the target nucleic acid, wherein said first part and said second part are bound via a nucleotide linker.

28. The process according to claim 27, wherein step a) comprises contacting at least two probes P1, wherein each of said probe P1 comprises or consists of: a nucleic acid fragment NA1, wherein each of said NA1 is designated as NA1-1 or NA1-2 and is respectively complementary to a nucleotide sequence T1-1 or T1-2 of the target nucleic acid.

29. The process according to claim 27, wherein step a) comprises contacting
i) at least two probes P3, comprising or consisting of: a first part comprising, or consisting of, a nucleic acid fragment NA4 having a nucleotide sequence complementary to the nucleotide sequence of NA2 and a second part comprising, or consisting of, a nucleic acid fragment NA3 having a nucleotide sequence complementary to the nucleotide sequence of a region T3 of the target nucleic acid, wherein said first part and said second part are bound via a nucleotide linker, and
ii) at least one probe P2 comprising, or consisting of, a nucleic acid fragment NA2 linked to an ultrabright luminescent particle.
